(19) United States
(12) Patent Application Publication
(10) Pub. No.: US 2022/0160658 A1
(43) Pub. Date: May 26, 2022

(54) TESOFENSINE FOR REDUCTION OF BODY WEIGHT IN PRADER-WILLI PATIENTS

(71) Applicant: Saniona A/S, Glostrup (DK)

(72) Inventors: Jørgen DREJER, Glostrup (DK); Thomas Amos JACOBSEN, Glostrup (DK); Thomas FELDTHUS, Glostrup (DK)

(21) Appl. No.: 17/420,181

(22) PCT Filed: Jan. 7, 2020
(86) PCT No.: PCT/EP2020/050153

§ 371 Date: Jul. 1, 2021

(30) Foreign Application Priority Data
Jan. 7, 2019 (CA) 3029052
Oct. 16, 2019 (CA) 3058933

(51) Int. Cl. A61K 31/138; A61K 45/06; A61K 31/404; A61K 31/46; A61K 9/20; A61K 9/24; A61K 9/50; A61P 3/04

(52) U.S. Cl. CPC A61K 31/138; A61K 45/06; A61K 31/404; A61P 3/04; A61K 9/2077; A61K 9/209; A61K 9/5078; A61K 31/46

(57) ABSTRACT

The present invention relates to a method of reducing body weight or hyperphagia in Prader-Willi patients comprising administering the active compound Tesofensine or a pharmaceutically acceptable salt thereof, preferably by the administration of a controlled release formulation comprising the active compounds tesofensine and a beta blocker. The invention further relates to pharmaceutical compositions comprising no more than 0.150 mg of Tesofensine, or a pharmaceutically acceptable salt thereof, and no more than 25 mg Metoprolol

### CROSS-REFERENCE TO RELATED APPLICATIONS

This application is the National Stage of International Patent Application No. PCT/EP2020/050153, filed Jan. 7, 2020 which claims the benefit of Canadian Patent Application No. 3,029,052, filed Jan. 7, 2019 and Canadian Patent Application No. 3,058,933, filed Oct. 16, 2019, the disclosures of which are incorporated herein by reference in their entireties for any and all purposes.

### TECHNICAL FIELD

The present invention relates to the use of Tesofensine for the reduction of body weight or the reduction of hyperphagia in patients suffering from Prader-Willi syndrome (PWS). The invention further relates to pharmaceutical compositions comprising no more than 0.150 mg of Tesofensine, or a pharmaceutically acceptable salt thereof, and no more than 25 mg Metoprolol.

### BACKGROUND

Prader-Willi syndrome or PWS is a complex genetic condition that affects many parts of the body. In infancy, this condition is characterized by weak muscle tone (hypotonia), feeding difficulties, poor growth, and delayed development. Beginning in childhood, affected individuals develop an insatiable appetite, which leads to chronic overeating (hyperphagia) and obesity. Therefore, weight reduction is critical for PWS patients suffering from obesity. Thus, there is impetus for finding new and alternative ways of treating and managing obesity resulting from PWS.

Tesofensine, i.e. [(1R,2R,3S,5S)-3-(3,4-dichlorophenyl)-2-(ethoxymethyl)-8-methyl-8-azabicyclo[3.2.1]octane], first described in WO 97/30997, is a triple monoamine reuptake inhibitor in development for the treatment of obesity.

Tesofensine effectively produces a weight loss in obese individuals of about twice of that seen with currently marketed anti-obesity drugs. Results from clinical studies with Tesofensine also showed that the compound has a good safety profile and is well tolerated. However, though no clinically relevant cardiovascular adverse events or changes in either blood pressure or pulse were seen, some cardiovascular effects were measured with slight increases in heart rate and trends in blood pressure. Although such small effects have no immediate risk to the patient, some medical and regulatory concerns have been raised based on observational studies, that even small changes in cardiovascular parameters may have long term implications on patients' benefit/risk evaluation.

Preclinical and clinical data suggest that appetite suppression is an important mechanism by which Tesofensine exerts its robust weight reducing effect. Notably, the strong hypophagic response (i.e. less appetite, decreased feeding) to Tesofensine treatment is demonstrated to be linked to central stimulation of noradrenergic and dopaminergic neurotransmission. However, the sympathomimetic mode of action of Tesofensine may also associate with the elevated heart rate and blood pressure observed in clinical settings.

Beta blockers, (β-blockers, beta-adrenergic blocking agents, beta antagonists, beta-adrenergic antagonists, beta-adrenoreceptor antagonists, or beta adrenergic receptor antagonists) are a class of drugs that are typically used for the management of cardiac arrhythmias, protecting the heart from a second heart attack (myocardial infarction) after a first heart attack (secondary prevention), and, in certain cases, hypertension. Beta blockers are also well known for their reductive effect on heart rate.

Metoprolol, i.e. 1-(Isopropylamino)-3-[4-(2-methoxyethyl)-phenoxy]-propan-2-ol, branded under various trade names, is a selective β1 (adrenergic) receptor blocker normally used in the treatment of various disorders of the cardiovascular system, and in particular hypertension.

Carvedilol ((±)-[3-(9H-carbazol-4-yloxy)-2-hydroxpropyl][2-(2-methoxyphenoxy)ethyl]amine) is a mixed, i.e. nonselective alpha and beta blocker. It is marketed under various trade names and is traditionally used in the treatment of mild to severe congestive heart failure (CHF) and high blood pressure.

WO 2013/120935 describes treatment of obesity by co-administration of tesofensine and metoprolol in order to ameliorate drug-induced elevation of blood pressure or increase in heart rate.

The serum half-life of tesofensine is nine days (Bara-Jimenez W, Dimitrova T, Sherzai A, Favit A, Mouradian M M, Chase T N (2004). “Effect of monoamine reuptake inhibitor NS 2330 in advanced Parkinson's disease”. Mov Disord 19 (10): 1183-6.). In comparison, the half-life of beta blockers is quite short with metoprolol in the order of 3-4 hours and carvedilol about 7 to 10 hours. Therefore simultaneous daily administration of these two drugs is likely to induce high fluctuations in the serum levels of the beta blocker and potentially recurrent temporary absence of therapeutic efficacy of the beta blocker.

### SUMMARY

The present inventors discovered an encouraging efficacy signal, particularly the effect on hyperphagia in a study on nine adult patients with PWS. Several patients discontinued prematurely due to adverse events. It is assumed that these adverse events were driven by unexpectedly high plasma concentrations of tesofensine Because of these observations the current inventors contemplate treatment of Prader-Willi patients at a daily dose of 0.250 mg or less.

Thus, the present invention relates to the use of tesofensine for reduction of body weight in patients with PWS.

In a first aspect, the present disclosure relates to a method of reduction of body weight or reduction of hyperphagia in Prader-Willi patients comprising administering Tesofensine or a pharmaceutically acceptable salt thereof in a daily dosage of 0.250 mg or less.

In a second aspect, the present disclosure relates to a method of maintaining body weight and preventing an increase in body weight.

In a third aspect, the present disclosure relates to a pharmaceutical composition comprising no more than 0.25 mg Tesofensine; and 5 to 100 mg of ER beta blocker, such as Metoprolol; and 1 to 25 mg of IR beta blocker, such as Metoprolol.

### DESCRIPTION OF DRAWINGS

FIG. 1: An overview of the number of patients from the two countries Hungary (HU) and Czech Republic (CZ), including treatment allocation, sex, age, weight at baseline and patients who completed the study can be seen in Table 7. The term “IMP” stands for Investigation Medicinal Product and corresponds to the co-administration of tesofensine (0.50 mg)/metoprolol (50 mg). FIG. 1a displays the change in body weight over time in kilograms, whereas FIG. 1b displays the change in body weight in %, calculated as ((body weight at visit 2, 5, 9 or 14)−(body weight at visit1))/(body weight at visit 1)×100. Visits were performed on days 1, 7, 28, 56 and 91 (visits 1, 2, 5, 9 and 14). The bars indicate standard deviations.

FIG. 2: The individual hyperphagia scores over time for individual patients receiving either the IMP (A) or the placebo (B).

FIG. 3: Change in waist circumference calculated at visit 2 (day 7), visit 5 (day 28), visit 9 (day 56) and visit 14 (day 91) by the following formula: change in waist circumference=(waist circumference at day 7, 28, 56, or 91 after start of treatment)−(waist circumference at baseline).

FIG. 4: The average tesofensine trough plasma concentrations from the TIPO-1 can be seen. With a dose of 0.5 mg Tesofensine (NS2330), a trough plasma concentration of 10 ng/mL was achieved.

FIG. 5: Comparison of weight loss in tesomet and placebo treated adult PWS patients from treatment initiation. Last observation carried forward if participating in study. The diagram shows mean and SEM.

FIG. 6: The results of the hyperphagia score in adult patients with PWS showed that food cravings fell from 10 (n=6) at baseline to 1 (n=5) after 56 days and to 0 (n=2), or no observation of hyperphagia, after 91 days in patients on Tesomet. Hyperphagia scores for patients on placebo varied over time (n=2) but did not change from baseline to day 91. The diagram shows mean and SEM.

FIG. 7: Body weight following Tesomet versus placebo treatment in adolescents with PWS (Example 4). A) Left part shows change (%) in body weight for all patients in the study; right part shows change (%) in body weight for the three patients who received 0.25 mg tesofensine in OLE2. B) The diagram shows the monthly change in body weight by plasma level in individual patients (each patient has a specific mark). Most of the patients were followed for 6 months or longer, and the body weight was measured on a monthly basis. Trend lines for each patient are disclosed in the diagram. The patients appear to lose weight when the plasma concentration is 7 to 11 ng/mL. C) Random coefficients analysis for the dataset in FIG. 7B. A strong, linear correlation is observed between tesofensine plasma level and monthly weight loss with a slope different from placebo (black line). Outer grey lines represent 95% prediction limit, and inner grey lines represent 95% confidence interval. (N=8; Intercept (SE)=2.932 (0.6566), p=0.0029; Slope (SE)=−0.460 (0.1120), p=0.0046; Covariance (Intercept, Slope)=−0.0710, Residual error=3.1208, AIC=214.72).

FIG. 8: BMI following Tesomet versus placebo treatment in adolescents with PWS. (Example 4). A) Left part shows change (%) in BMI for all patients in the study; right part shows change (%) in BMI for the three patients who received 0.25 mg tesofensine in OLE2. B) The diagram shows the monthly change in BMI by plasma level in individual patients (each patient has a specific mark). Most of the patients were followed for 6 months or longer, and the BMI was measured on a monthly basis. Trend lines for each patient are disclosed in the diagram. C) Random coefficients analysis for the dataset in FIG. 8B. A strong, linear correlation is observed between tesofensine plasma level and monthly reduction in BMI with a slope different from placebo (black line). Outer grey lines represent 95% prediction limit, and inner grey lines represent 95% confidence interval. (N=8; Intercept (SE)=2.529 (0.617), p=0.0040; Slope (SE)=−0.478 (0.1081), p=0.0031; Covariance (Intercept, Slope)=−0.0570, Residual error=2.9875, AIC=214.53). Last observation carried forward if participating in study. For height data in relation to BMI, linear regression if data available

FIG. 9: Hyperphagia score following Tesomet versus placebo treatment in adolescents. The diagram in A) shows all patients, and the diagram in B) shows the four patients who completed OLE2. Last observation carried forward if participating in study. The data show mean and SEM.

### Definitions

Extended release−ER—also known as sustained-release [SR], extended-release [ER, XR, XL], and controlled-release [CR], is a mechanism used in pill tablets or capsules to dissolve a drug over time in order to be released slower and steadier into the bloodstream.

Immediate release—IR. The drug is released (dissolved) immediately after ingestion.

### DETAILED DESCRIPTION

Treatment of Prader-Willi Patients

Disclosed herein is a method of reduction of body weight or reduction of hyperphagia in Prader-Willi patients comprising administering Tesofensine or a pharmaceutically acceptable salt thereof in a daily dosage of less than 0.250 mg. I.e., in one aspect, the present invention relates to a pharmaceutical composition comprising Tesofensine, or a pharmaceutically acceptable salt thereof, for use in the treatment hyperphagia and/or for use in reduction or maintenance of body weight in Prader-Willi patients, wherein Tesofensine is administered in a daily dosage of 0.250 mg or less. In one aspect, the present application relates to Tesofensine, or a pharmaceutically acceptable salt thereof, for use in a method of reduction or maintenance of body weight and/or reduction of hyperphagia in Prader-Willi patients.

In one embodiment of the invention, Tesofensine is administered in combination with a beta blocker.

The composition as described herein is useful as a medicament, e.g. for the reduction or maintenance of body weight or reduction of hyperphagia in Prader-Willi patients, i.e. patients suffering from Prader-Willi syndrome (PWS). In one embodiment, said Prader-Willi patient is an adult. In one embodiment, said Prader-Willi patient is an adolescent.

Prader-Willi syndrome (PWS) is a genetic condition that affects many parts of the body. Infants with PWS have severe hypotonia (low muscle tone), feeding difficulties, and slow growth. In later infancy or early childhood, affected children typically begin to eat excessively and become obese. Other signs and symptoms often include short stature, hypogonadism, developmental delays, cognitive impairment, and distinctive behavioral characteristics such as temper tantrums, stubbornness, and obsessive-compulsive tendencies. PWS is caused by missing or non-working genes on chromosome 15. Most cases are not inherited and occur randomly. Rarely, a genetic change responsible for PWS can be inherited.

Prader-Willi syndrome is characterized by severe infantile hypotonia with poor suck and failure to thrive; hypogonadism causing genital hypoplasia and pubertal insufficiency; characteristic facial features; early-childhood onset obesity and hyperphagia; developmental delay/mild intellectual disability; short stature; and a distinctive behavioral phenotype. Sleep abnormalities and scoliosis are common. Growth hormone insufficiency is frequent, and replacement therapy provides improvement in growth, body composition, and physical attributes. Management is otherwise largely supportive.

Consensus clinical Prader-Willi diagnostic criteria exist, but diagnosis should be confirmed through genetic testing. Prader-Willi syndrome is due to absence of paternally expressed imprinted genes at 15q11.2-q13 through paternal deletion of this region (65-75% of individuals), maternal uniparental disomy 15 (20-30%), or an imprinting defect (1-3%). Parent-specific DNA methylation analysis will detect >99% of individuals. However, additional genetic studies are necessary to identify the molecular class. There are multiple imprinted genes in this region, the loss of which contribute to the complete phenotype of Prader-Willi syndrome. However, absence of a small nucleolar organizing RNA gene, SNORD116, seems to reproduce many of the clinical features. Sibling recurrence risk is typically <1%, but higher risks may pertain in certain cases. Prenatal diagnosis is available.

The term “hyperphagia” is used to express excessive hunger or desire for food often resulting in “overeating”. It can be caused by disorders such as diabetes, Kleine-Levin syndrome (a malfunction in the hypothalamus) and the genetic disorder Prader-Willi syndrome. There are several approaches frequently used to describe hyperphagia:
By quantifying “overeating” as energy intake relative to a control group; eating beyond amount predicted for body size and body composition; and evaluating food intake pre- vs. post-treatment (e.g., before and after people with leptin deficiency are given recombinant leptin);
Relative to a control group, by evaluating “hunger” (e.g., with visual analog scales in patients with PWS and controls); time to reach satiation relative to a control group; and duration of satiety;
Measuring preoccupation with food or “hyperphagic drive”; food seeking behaviors (e.g., night eating, etc.); and
Evaluating psychological symptoms such as distress and functional impairment.

Clinical assessment of hyperphagia is done using a “Hyperphagia Questionnaire for Clinical Trials (HQ-CT)” as described by Fehnel et al. in “Development of the Hyperphagia Questionnaire for Use in Prader-Willi Syndrome Clinical Trials” at the ISPOR 20th Annual International Meeting.

Due to the particular combination of extended and immediate release forms of a beta blocker in combination with tesofensine as described herein the composition of the present disclosure effectively alleviates cardiovascular side-effects of tesofensine while maintaining the therapeutic efficacy of tesofensine.

In one embodiment the method of the present disclosure is useful for the reduction of body weight or hyperphagia in Prader-Willi patients.

In one embodiment, the method of the present disclosure reduces body weight of the Prader-Willi patient by at least 3% after two months of treatment, such as between 5% and 10% or between 3% and 6%.

In one embodiment, the method of the present disclosure results in a reduction of the waist circumference of the Prader-Willi patient by at least 4 cm after 56 days of treatment, such as between 4 and 6 cm or between 6 and 10 cm.

For adolescent PWS patients the aim is not necessarily to reduce body weight, because as adolescents they need to grow. However, the craving of adolescent PWS patients is often so uncontrolled that they gain too much weight in form of fat. Therefore, for adolescents controlling the body weight to prevent weight gain is often desired. In one embodiment, the method of the present disclosure is a method for maintenance of body weight. In one embodiment, the method of the present invention is a method for reducing the increase in body weight, e.g. preventing an increase in body weight.

Body Mass Index (BMI) is a value derived from the mass (weight) and height of a person. The BMI is defined as the body mass divided by the square of the body height, and is universally expressed in units of kg/m². In one aspect, the present disclosure relates to a method for reducing or maintaining BMI in Prader-Willi patients.

In one embodiment, the method according to the present disclosure is used for reduction or maintenance of body weight or hyperphagia in Prader-Willi patients suffering from hypothalamic obesity (HO). HO typically in occurs in patients with tumors and lesions in the medial hypothalamic region. Hypothalamic dysfunction can lead to hyperinsulinemia and leptin resistance. These patients have often suffered damage to the hypothalamus. Damage to the hypothalamus has long been known to promote excessive eating (hyperphagia) and weight gain, termed “hypothalamic obesity.” This form of weight gain is often not responsive to diet and exercise.

In one embodiment the composition of the present disclosure is for use in a method of decreasing liver fat and/or visceral adiposity. Reduction of liver fat and/or visceral adiposity has been shown to be effective in the treatment of fatty liver disorders. Tesofensine significantly decreases waist circumference and sagittal diameter (Astrup et al., 2008, Lancet 372: 1906-13); hence tesofensine is capable of reducing visceral adiposity.

The method of the present disclosure is preferably administered to a subject in need thereof once a day. However, in certain embodiments, the composition may be administered more than once a day, such as twice a day or alternatively less than once a day, such as once every second or third day depending on the specific formulation and concentration of the individual components of the composition. The subject treated is preferably a human, such as an adult human aged 18 or older.

In one embodiment the present disclosure relates to use of the composition as disclosed herein in the manufacture of a medicament for the reduction or maintenance of body weight or reduction of hyperphagia in Prader-Willi patients.

In one embodiment, the method of the present disclosure results in a steady state Tesofensine plasma concentration of from 2 to 15 ng/mL, such as 5 to 15 ng/mL.

In one embodiment, the method of the present disclosure is for maintenance of body weight wherein the dosage results in a steady state Tesofensine plasma concentration of 3 to 6 ng/mL. In one embodiment, the dosage resulting in a steady state Tesofensine plasma concentration of 3 to 6 ng/mL is about 0.125 mg Tesonfensine. In one embodiment, the method of the present disclosure is a method for maintaining body weight in adolescent Prader-Willi patients, said method comprising administering Tesofensine or a pharmaceutically acceptable salt thereof in a daily dosage of about 0.125 mg. This is demonstrated in Example 4 of the present disclosure.

In one embodiment, the method of the present disclosure is for reduction of body weight wherein the dosage results in a steady state Tesofensine plasma concentration of 7 to 11 ng/mL. In one embodiment, the dosage resulting in a steady state Tesofensine plasma concentration of 7 to 11 ng/mL is about 0.250 mg Tesonfensine. In one embodiment, the method of the present disclosure is a method for reducing body weight in adolescent Prader-Willi patients, said method comprising administering Tesofensine or a pharmaceutically acceptable salt thereof in a daily dosage of about 0.250 mg. This is demonstrated in Example 4 of the present disclosure.

In one embodiment, the method of the present disclosure is for reduction of hyperphagia wherein the dosage results in a steady state Tesofensine plasma concentration of 7 to 11 ng/mL. In one embodiment, the dosage resulting in a steady state Tesofensine plasma concentration of 7 to 11 ng/mL is about 0.250 mg Tesonfensine. In one embodiment, the method of the present disclosure is a method for reducing hyperphagia in adolescent Prader-Willi patients, said method comprising administering Tesofensine or a pharmaceutically acceptable salt thereof in a daily dosage of about 0.250 mg. This is demonstrated in Example 4 of the present disclosure.

In an aspect of the invention, the method of reducing body weight or reducing hyperphagia in Prader Willi patients comprises:
a. a first composition comprising an extended release (ER) composition of an active pharmaceutical ingredient (API) selected from the beta-blocker or a pharmaceutically acceptable salt thereof,
b. a second composition comprising an active pharmaceutical ingredient (API) selected from Tesofensine or a pharmaceutically acceptable salt thereof, and
c. a third composition comprising an immediate release (IR) composition of an active pharmaceutical ingredient (API) selected from a beta blocker or a pharmaceutically acceptable salt thereof.

The term “Tesomet” as used herein relates to combination treatment of tesofensine and metoprolol.

Pharmaceutical Composition

In one aspect, the present invention relates to a pharmaceutical composition comprising no more than 0.150 mg of Tesofensine, or a pharmaceutically acceptable salt thereof, and no more than 25 mg Metoprolol.

In one embodiment, the Metoprolol comprises an extended release composition of Metoprolol, or a pharmaceutically acceptable salt thereof, (ER Metoprolol); and an immediate release composition of Metoprolol, or a pharmaceutically acceptable salt thereof (IR Metoprolol).

In one embodiment, the pharmaceutical composition comprises:
a. a first composition comprising an extended release (ER) composition of an active pharmaceutical ingredient (API) selected from the beta-blocker or a pharmaceutically acceptable salt thereof,
b. a second composition comprising an active pharmaceutical ingredient (API) selected from Tesofensine or a pharmaceutically acceptable salt thereof, and
c. a third composition comprising an immediate release (IR) composition of an active pharmaceutical ingredient (API) selected from a beta blocker or a pharmaceutically acceptable salt thereof.

In one aspect, the invention concerns a pharmaceutical composition comprising said first composition, second composition and third composition. In one embodiment, said pharmaceutical composition comprises no more than 0.250 mg of Tesofensine, or a pharmaceutically acceptable salt thereof; and 5 to 100 mg of ER beta blocker, such as Metoprolol; and 1 to 25 mg of IR beta blocker, such as Metoprolol.

The beta blocker may for example be metoprolol or carvedilol or pharmaceutically acceptable salts thereof. These include the phosphate, succinate, maleate, sulfate, glutarate, lactate, benzoate, and mandelate salts.

The in vitro bio-dissolution profile (as determined by USP Type II apparatus, rotating paddle, with 500 mL of Phosphate buffer at pH 7.4, 37° C. set at rotating speed of 50 rpm) of the beta blocker is preferably as in table 1.

TABLE 1
In vitro bio-dissolution profile of
extended release beta blocker.

| Dissolution time | Range |
|---|---|
| 1 hour | 10-35% |
| 4 hours | 25-45% |
| 8 hours | 45-65% |
| 20 hours | >80% |

For example, the combined in vitro bio-dissolution profile of metoprolol preferably has a dissolution profile lying within one or more of the release ranges in table 2 for different metoprolol IR:ER ratios at various time points (as determined by USP Type II apparatus, rotating paddle, with 900 mL of Phosphate buffer at pH 7.4, 37° C. set at rotating speed of 75 rpm).

TABLE 2
Combined in vitro bio-dissolution profile of metoprolol.

| | Calculated | | Calculated | | |
|---|---|---|---|---|---|
| | dissolution | | dissolution | | |
| | 10 mg | Dissolution | 25 mg | Dissolution | |
| Dissolution | IR + 100 mg | ranges | IR + 100 mg | ranges | Overall |
| time | ER | (10:100) | ER | (25:100) | range |
| 1 hour | 13% | 10-20% | 23% | 20-30% | 10-30% |
| 4 hours | 29% | 20-40% | 38% | 30-50% | 20-50% |
| 8 hours | 53% | 40-65% | 58% | 50-70% | 40-70% |
| 24 hours | 88% | >80% | 90% | >80% | >80% |
| 1 hour | 13% | 10-20% | 23% | 20-30% | 10-30% |
| 4 hours | 29% | 20-40% | 38% | 25-50% | 20-50% |
| 8 hours | 53% | 40-65% | 58% | 40-70% | 40-70% |
| 20 hours | 88% | >80% | 90% | >80% | >80% |

In general the tesofensine of the composition is dissolved within ½-1 hour. The in vitro dissolution profile with tesofensine under the conditions above is at least 80% of the API within 45 minutes.

Many physiological factors influence both the gastrointestinal transit time and the release of a drug from a controlled release dosage form, and thus influence the uptake of the drug into the systemic circulation. A sustained-release dosage form should release the beta blocker at a controlled rate such that the amount of active ingredient available in the body to treat the condition is maintained at a relatively constant level over an extended period of time. The release of an active ingredient from a controlled release dosage form is generally controlled by diffusion through a coating.

It is likewise important that part of the beta blocker is released rapidly so that a therapeutically effective level of the beta blocker is reached rapidly.

In one embodiment, the pharmaceutical composition is in form of a pharmaceutical dosage form, such as a tablet or a capsule. In one embodiment, the pharmaceutical composition is formulated as a dosage unit. In one embodiment, the pharmaceutical composition is formulated as a once daily dosage unit.

Tesofensine

The pharmaceutical composition described herein comprises an active pharmaceutical ingredient (API) selected from tesofensine or a pharmaceutically acceptable salt thereof.

Tesofensine [(1R,2R,3S,5S)-3-(3,4-dichlorophenyl)-2-(ethoxymethyl)-8-methyl-8-azabicyclo[3.2.1]octane] is a centrally acting triple monoamine re-uptake inhibitor (MRI) with intrinsic inhibitory activity on noradrenaline, serotonin and dopamine transporter function. When corrected for placebo and diet effects, long-term Tesofensine treatment produces a weight loss of about 10% in obese patients, which is twice as much as that achieved by currently marketed anti-obesity drugs.

The chemical structure of Tesofensine is:

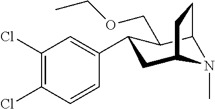

Preclinical and clinical data suggest that appetite suppression is an important mechanism by which Tesofensine exerts its robust weight-reducing effect. In addition, Tesofensine has also been demonstrated to increase nocturnal energy expenditure in human subjects. These findings have recently been corroborated and extended in preclinical settings, demonstrating that Tesofensine induces a robust and sustained weight loss in a rat model of diet-induced obesity (DIO) of which the long-lasting reduction in body weight is caused by appetite suppression with a gradual increase in energy expenditure. Notably, the hypophagic effect of Tesofensine in DIO rats is critically dependent on stimulated α1 adrenoceptor activity, and to a less extend dopamine D1 receptor function, indicating that enhancement of central noradrenergic and dopaminergic neurotransmission constitute important mechanisms underlying the robust appetite-suppressing effect of Tesofensine.

Overall, chronic Tesofensine treatment is associated with minor adverse events, and with minimal cardiovascular effects, suggesting that Tesofensine may generally be a well-tolerated long-term treatment for obesity. However, dose-dependent elevations in heart rate and significant increases in blood pressure have been reported in obese individuals. The long-term implications of such Tesofensine-induced cardiovascular effects are not known and can potentially play a role in the benefit/risk evaluation of patients treated with Tesofensine.

Beta Blockers

The present invention involves the use of beta blockers in certain embodiments. The beta blocker may be any conventional beta blocker known in the art. Preferably, the beta blocking drug is selected from the following groups of compounds, which groups of compounds are known in the art and may be commercially available under different brand names, or may be obtained as described in the literature.

In one embodiment, the beta blocker in the ER composition is the same beta blocker as in the IR composition.

In one embodiment, the pharmaceutical composition comprises ER Metoprolol and IR Metoprolol.

As used herein, the term “ER Metoprolol” refers to an extended release (ER) composition of Metoprolol, or a pharmaceutically acceptable salt thereof.

As used herein, the term “IR Metoprolol” refers to an immediate release (IR) composition of Metoprolol, or a pharmaceutically acceptable salt thereof.

Non-Selective Beta Blockers

In one embodiment, the beta blocker is a non-selective beta blocker. Examples of non-selective beta blockers include alprenolol, amosulalol, bucindolol, carteolol, levobunolol, mepindolol, metipranolol, nadolol, oxprenolol, penbutolol, pindolol, propranolol, sotalol and timolol.

In one embodiment, the beta blocker is selected from the group consisting of alprenolol, amosulalol, bucindolol, carteolol, levobunolol, mepindolol, metipranolol, nadolol, oxprenolol, penbutolol, pindolol, propranolol, sotalol, timolol and pharmaceutically acceptable salts thereof.

Beta 1-Selective Beta Blockers

In another embodiment, the beta blocker is a beta 1-selective beta blocker.

Examples of beta 1-selective beta blockers include acebutolol, atenolol, betaxolol, bisoprolol, esmolol, landiolol, metoprolol and nebivolol.

In one embodiment, the beta blocker is selected from the group consisting of acebutolol, atenolol, betaxolol, bisoprolol, esmolol, landiolol, metoprolol, nebivolol and pharmaceutically acceptable salts thereof.

In a particular embodiment, the beta blocker is metoprolol or a pharmaceutically acceptable salt thereof.

Mixed Alpha and Beta Blockers

In a still further embodiment, the beta blocker is a mixed alpha and beta blocker.

Examples of mixed alpha and beta blockers include carvedilol, celiprolol and labetalol.

In one embodiment, the beta blocker is selected from the group consisting of carvedilol, celiprolol, labetalol and pharmaceutically acceptable salts thereof.

In a particular embodiment, the beta blocker is carvedilol or a pharmaceutically acceptable salt thereof.

Beta 2-Selective Beta Blockers

In a still further embodiment, the beta blocker is a beta 2-selective beta blocker.

One example of a beta 2-selective beta blocker is butaxamine.

In one embodiment, the beta blocker is butaxamine or a pharmaceutically acceptable salt thereof.

Pharmaceutically Acceptable Salts

Examples of pharmaceutically acceptable salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzene-sulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of an API include, without limitation, the sodium, the potassium, the calcium, the magnesium, the zinc, the aluminium, the lithium, the choline, the lysinium, and the ammonium salt, and the like, of an API containing an anionic group. Such cationic salts may be formed by procedures well known and described in the art.

In the context of this disclosure the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

In one embodiment of the present disclosure, Tesofensine is selected from the free base, the citrate salt and the tartrate salt.

Suitable pharmaceutically acceptable salts of metoprolol include any of the salts mentioned herein and preferably include the tartrate, succinate, fumarate or benzoate salts and especially the succinate salt. The S-enantiomer of metoprolol or a salt thereof, particularly the benzoate salt or the sorbate salt, may also be used.

Similarity Factors

Similarity factor (f2) is a recognized method for the determination of the similarity between the dissolution profiles of a reference and a test compound. Similarity factor (f2) is a logarithmic transformation of the sum of squared error. The similarity factor (f2) is 100 when the test and reference profiles are identical and approaches zero as the dissimilarity increases. The similarity factor has also been adapted to apply to the determination of the similarity between the dissolution profiles of a reference and test compound as they relate to modified release formulations, such as those exemplified herein.

The f2 similarity factor has been adopted in the SUP AC guidelines and by the FDA guidance on dissolution testing of immediate release dosage forms (FDA Guidance for Industry, Dissolution Testing of Immediate Release Solid Oral Dosage Forms, FDA, (CDER), August 1997 (Dissolution Tech. 4, 15-22, 1997)).

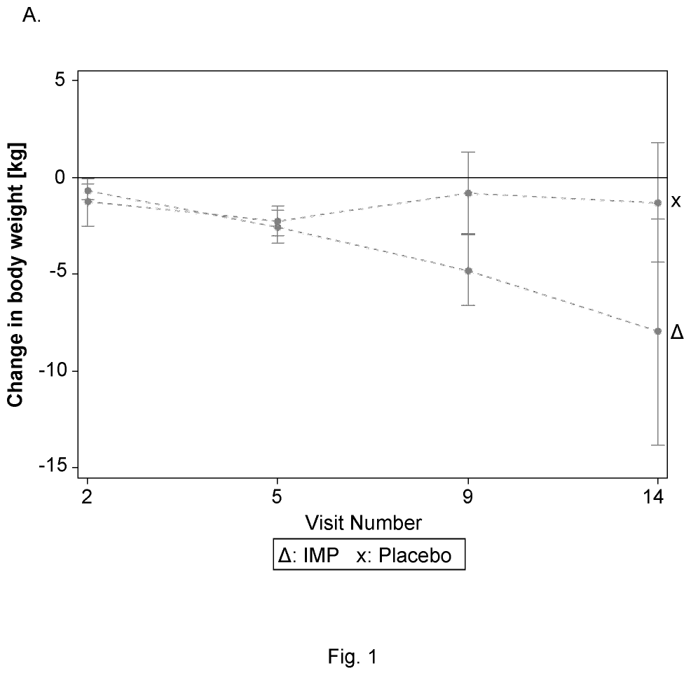

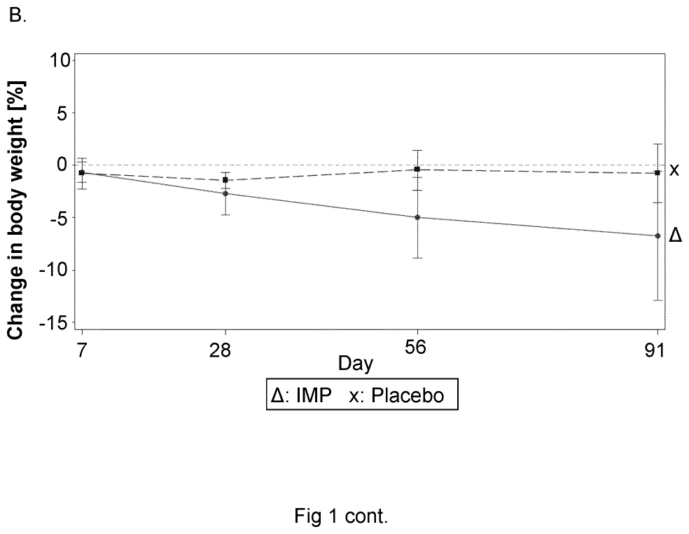

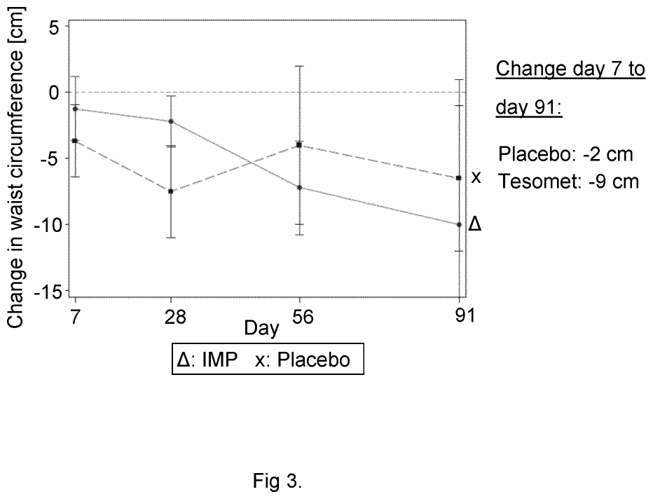

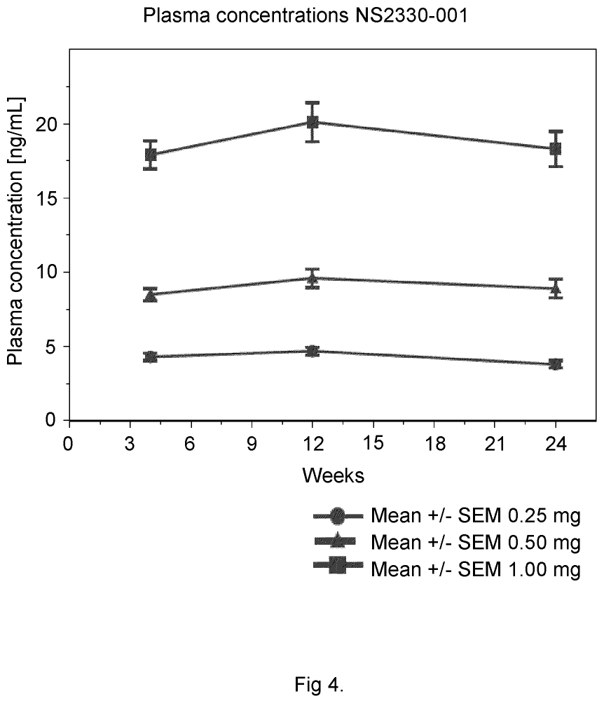

Preferably the pharmaceutical composition has a beta blocker in vitro dissolution profile generated using the USP Type II apparatus, rotating paddle method as described herein with a similarity factor (f2) between 50 and 100 when calculated using one of the examples from FIG. 1 or FIG. 3 as the reference profile.

API Amounts and Ratios in Pharmaceutical Composition

Amount of Beta Blocker

In one embodiment, the pharmaceutical composition as used herein comprises a beta-blocker, or a pharmaceutically acceptable salt thereof. In one embodiment, the pharmaceutical composition as used herein is administered in combination with a beta-blocker, or a pharmaceutically acceptable salt thereof.

The ratio of extended release beta blocker, such as metoprolol, to immediate release beta blocker may be 75-95:25-5. Suitably, the beta blocker, such as metoprolol, in a pharmaceutical composition is approximately an 80:20 ratio of extended release to immediate release amounts, i.e. the ratio of ER Metoprolol/IR Metoprolol is about 4:1 by weight. In another embodiment, the beta blocker, such as metoprolol, is in an approximate 90:10 or 100:10 ratio of extended to immediate release amounts. In still another embodiment, the ratio is approximately 80:20 or 75:25. Explained differently, for a unit dosage form, such as a tablet, containing 40 mg beta blocker, such as metoprolol, the beta blocker may be present in an amount of about 30 mg in the extended release phase and about 10 mg in the immediate release phase. For a unit dosage form comprising 22 mg beta blocker, such as metoprolol, the beta blocker ER may be present in an amount of 20 mg and the beta blocker IR may be present in an amount of 2 mg. For example, in one embodiment, the ratios of extended release to immediate release phase represent the proportional amount of each layer in a bi-layer dosage form. In another embodiment, the ratios represent the amount of metoprolol in the extended release intragranular component versus the immediate release extragranular component of a single layer dosage form. The ratios and amounts mentioned in the current paragraph apply well to metoprolol as the beta-blocker.

In one embodiment, one dosage form comprises an amount of beta blocker, such as metoprolol, of no more than 100 mg, such as about 75 mg, such as about 50 mg, such as about 25 mg such as about 12.5 mg beta blocker.

Preferably one dosage form comprises an amount of beta blocker, such as metoprolol, ER of 5-200 mg, such as 25-200 mg, such as 5-100 mg API, such as 15-100 mg of API, preferably 15-50 mg, such as 15-40 mg, such as 5-50 mg, such as 5-20 mg, for example about 8 mg, about 20 mg or about 40 mg. In one embodiment, one dosage form comprises an amount of beta blocker, such as metoprolol, ER of no more than 200 mg API, such as no more than 150 mg, such as no more than 100 mg, such as no more than 50 mg, such as no more than 20 mg.

In one embodiment, one dosage form comprises an amount of beta blocker, such as metoprolol, ER of no more than 80 mg, such as about 60 mg, such as about 40 mg, such as about 20 mg, such as about 10 mg.

Other beta-blockers may require lower dosages. In this case one dosage form may comprise an amount of beta blocker, such as carvedilol, ER of 5-40 mg of API, such as 10-20 mg of API, preferably 12-20, for example about 15 mg.

The amount of beta blocker, such as metoprolol, IR per dosage form may be from 1-25 mg API, such as 1-15 mg, for example 3-15 mg, such as 4-10 mg, such as 5-10 mg, such as 1-10 mg, such as 1-5 mg, such as 2-5 mg, for example about 2 mg, about 5 mg, about 10 mg, about 6 mg, or about 8 mg. In one embodiment, the amount of beta blocker, such as metoprolol, IR per dosage form is no more than 25 mg API, such as no more than 20 mg, such as no more than 15 mg, such as no more than 10 mg, such as no more than 5 mg.

In one embodiment, one dosage form comprises an amount of beta blocker, such as metoprolol, IR of no more than 20 mg, such as about 15 mg, such as about 10 mg, such as about 5 mg, such as about 2.5 mg.

The amount of beta blocker as specified herein is based on an the amount of metoprolol tartrate. Other beta blockers, or pharmaceutically acceptable salts thereof, as well as other pharmaceutically relevant salts of metoprolol, or the free base, may also be used in amounts equivalent to the doses of metoprolol tartrate disclosed herein.

In one embodiment, the amount of ER Metoprolol in the pharmaceutical composition is in the range of 1 to 20 mg. In one embodiment, the amount of ER Metoprolol is in the range of 5 to 15 mg. In one embodiment, the amount of ER Metoprolol is 10 mg.

In one embodiment, the amount of IR Metoprolol in the pharmaceutical composition is in the range of 1 to 10 mg. In one embodiment, the amount of IR Metoprolol is in the range of 1 to 5 mg. In one embodiment, the amount of IR Metoprolol is 2.5 mg.

In one embodiment, the combined daily dosis of the beta-blocker are below 125 mg, such as between 5 and 50 mg, between 10 and 30 mg, for example 25 mg, or 12.5 mg. In one embodiment, the combined daily dosis of Metoprolol is less than 25 mg.

Amount of Tesofensine

The amount of tesofensine per dosage form (such as in the second composition) is generally between 0.010-0.250 mg API, between 0.01-0.250 mg, for example 0.025-0.200 mg, such as 0.040-0.125, such as 0.040-0.120 mg for example about 0.050 mg, 0.075 mg, 0.100 mg, 0.125 mg or 0.250 mg. In one embodiment, the amount of tesofensine per dosage form is no more than 0.25 mg API, such as no more than 0.250 mg, such as no more than 0.200 mg, such as no more than 0.150 mg, such as no more than 0.125 mg. The dose of Tesofensine is based on the amount of the free base, but pharmaceutically relevant salts of Tesofensine may also be used in amounts equivalent to the doses of the free base disclosed herein.

In one embodiment, the dosage of Tesofensine is between 0.125 and 0.250 mg.

In one embodiment, the amount of Tesofensine, or a pharmaceutically acceptable salt thereof, is in the range of 0.025 to 0.150 mg. In one embodiment, the amount of Tesofensine, or a pharmaceutically acceptable salt thereof, is in the range of 0.050 to 0.150 mg. In one embodiment, the amount of Tesofensine, or a pharmaceutically acceptable salt thereof, is in the range of 0.100 to 0.150 mg. In one embodiment, the amount of Tesofensine, or a pharmaceutically acceptable salt thereof, is about 0.125 mg, such as 0.125 mg.

Amounts of Tesofensine and Beta Blocker

In one embodiment, the ratio of the amount beta blocker, such as metoprolol, to tesofensine is about 200:1. In one embodiment, the ratio of the amount beta blocker, such as metoprolol, to tesofensine is about 100:1. In one embodiment, the ratio of Tesofensine/Metoprolol is about 1:100 by weight.

One dosage form may comprise 5-100 mg ER beta blocker, such as metoprolol, 2-25 mg IR beta blocker, such as metoprolol, and 0.010-0.250 mg tesofensine; for example 5-50 mg ER beta blocker, such as metoprolol, 2-15 mg IR beta blocker, such as metoprolol, and 0.025-0.250 mg tesofensine; for example 5-40 mg ER beta blocker, such as metoprolol, 2-10 mg IR beta blocker, such as metoprolol, and 0.025-0.075 tesofensine; for example 5-40 mg ER beta blocker, such as metoprolol, 2-10 mg IR beta blocker, such as metoprolol, and 0.125-0.250 mg tesofensine; for example 20-40 mg ER beta blocker, such as metoprolol, 5-10 mg IR beta blocker, such as metoprolol, and 0.050-0.250 tesofensine; for example 30-60 mg ER beta blocker, such as metoprolol, 8-15 mg IR beta blocker, such as metoprolol, and 0.050-0.250 tesofensine; for example 5-20 mg ER beta blocker, such as metoprolol, 2-5 mg IR beta blocker, such as metoprolol, and 0.125-0.250 tesofensine; for example 5-20 mg ER beta blocker, such as metoprolol, 2-15 mg IR beta blocker, such as metoprolol, and 0.025-0.250 tesofensine; for example 5-20 mg ER beta blocker, such as metoprolol, 2-5 mg IR beta blocker, such as metoprolol, and 0.025-0.150 tesofensine.

In one embodiment, the dosage form comprises 25-200 mg ER metoprolol, 5-50 mg IR metoprolol, and 0.250 mg Tesofensine or less, for example 50-125 mg ER metoprolol, 10-25 mg IR metoprolol, and 0.025-0.100 mg tesofensine, for example 75-80 mg ER metoprolol, 10-15 mg IR metoprolol, and 0.025-0.175 tesofensine.

In one embodiment, the dosage form comprises 5-200 mg ER metoprolol, 1-50 mg IR metoprolol, and 0.250 mg Tesofensine or less; for example 5-125 mg ER metoprolol, 1-25 mg IR metoprolol, and 0.025-0.125 mg tesofensine, for example 5-20 mg ER metoprolol, 1-10 mg IR metoprolol, and 0.025-0.175 tesofensine.

In one embodiment, one dosage form comprises 8-20 mg ER beta blocker, such as metoprolol, and 2-5 mg IR beta blocker, such as metoprolol and tesofensine. In one embodiment, one dosage form comprises 8-20 mg ER beta blocker, such as metoprolol, and 2-5 mg IR beta blocker, such as metoprolol and no more than 0.25 mg tesofensine. In one embodiment, one dosage form comprises about 10 mg ER beta blocker, such as metoprolol, about 2.5 mg IR beta blocker, such as metoprolol, and about 0.125 mg tesofensine. In one embodiment, one dosage form comprises about 20 mg ER beta blocker, such as metoprolol, about 5 mg IR beta blocker, such as metoprolol, and about 0.25 mg tesofensine. In one embodiment, one dosage form comprises about 30 mg ER beta blocker, such as metoprolol, about 7.5 mg IR beta blocker, such as metoprolol, and about 0.375 mg tesofensine. In one embodiment, one dosage form comprises about 40 mg ER beta blocker, such as metoprolol, about 10 mg IR beta blocker, such as metoprolol, and about 0.5 mg tesofensine.

In one embodiment, the dosage form comprises 8 mg of ER metoprolol; 2 mg of IR metoprolol; and 0.125 mg tesofensine. In one embodiment, the dosage form comprises 20 mg of ER metoprolol; 5 mg of IR metoprolol; and 0.250 mg tesofensine.

In one embodiment, the pharmaceutical composition comprises no more than 0.150 mg of Tesofensine, or a pharmaceutically acceptable salt thereof, no more than 20 mg of ER Metoprolol; and no more than 5 mg of IR Metoprolol. In one embodiment, the pharmaceutical composition comprises no more than 0.150 mg of Tesofensine, or a pharmaceutically acceptable salt thereof, 5 to 20 mg ER Metoprolol, and 1 to 5 mg IR Metoprolol.

In one embodiment, the pharmaceutical composition comprises 0.125 mg Tesofensine, or a pharmaceutically acceptable alt thereof, and 12.5 mg Metoprolol.

In one embodiment, the pharmaceutical composition comprises about 0.125 mg Tesofensine; about 10 mg ER Metoprolol and about 2.5 mg IR Metoprolol.

In one embodiment the beta blocker is metoprolol and the amount of the two APIs in the three phases of the current dosage form are present in the absolute amounts of table 3.

TABLE 3
Amounts of Metoprolol ER,
Metoprolol IR and Tesofensine.

| Metoprolol ER | Metoprolol IR | Tesofensine |
|---|---|---|
| 20-150 mg | 5-50 mg | 0.010-0.250 mg |
| 20-100 mg | 5-25 mg | 0.010-0.150 mg |
| 30-80 mg | 5-20 mg | 0.025-0.080 mg |
| 5-50 mg | 1-10 mg | 0.100-0.250 mg |
| 5-50 mg | 1-10 mg | 0.075-0.150 mg |
| 80 mg | 20 mg | 0.025-0.250 mg |
| 40 mg | 10 mg | 0.250 mg |
| 20 mg | 5 mg | 0.050 mg |

Multi-Layer Dosage Form

The extended release phase may be part of a multiple layer tablet, such as a bi or tri-layer dosage form.

In one embodiment, the dosage form comprises a tri-layer dosage unit having an extended release (ER) phase layer with a beta blocker, such as metoprolol or carvedilol, and one immediate release phase layer with a beta blocker, such as metoprolol or carvedilol and another immediate release layer with tesofensine. The ER phase contains a therapeutically effective amount of the beta blocker, such as metoprolol or carvedilol, suitably in granulate form.

In other embodiments, the dosage form is a bi-layer tablet having an ER phase layer with a beta blocker, such as metoprolol or carvedilol and one immediate release layer with both the betablocker (such as metoprolol or carvedilol) and tesofensine.

Extended Release Phase

Extended release compositions of beta blockers, such as metoprolol or pharmaceutically acceptable salts of metoprolol are known the art. Non-limiting examples of disclosures of such compositions are found in: WO 2015/004617, WO 2013/084089, WO 2013/030725, WO 2012/052834, WO 2011/143420, WO 2007/09770, WO 2004/069234, WO 2007/110753, WO 2007/029070, WO 2008/012346, and WO 2007/048233. Such extended release compositions typically involve coating the API with an extended release layer that provides an approximated zero-order rate of dissolution of the API.

In one embodiment, the extended release beta blocker, such as metoprolol, is formulated as pellets with pharmaceutically acceptable excipients such as for example binders, film coating polymers, plasticizers, starch, glidants, and disintegrants.

An extended release formulation of carvedilol is also known from U.S. Pat. No. 8,101,209 (Flamel Technologies).

Inert Core

In some embodiments, the pellets comprise an initial core (inert core) coated with a layer of a beta blocker, such as metoprolol or a metoprolol salt, and further coated with an extended release layer.

As used herein the term initial core refers to a pharmaceutically acceptable core for use in pharmaceutical formulations which core is inert.

In one embodiment there is provided a pharmaceutical composition for extended release comprising pellets coated with a beta blocker, such as metoprolol or a metoprolol salt, wherein each coated pellet comprises a) an inert core comprising at least 50% (w/w) of soluble substance; b) a drug layer comprising the beta blocker, such as metoprolol, which layer covers the inert core; and c) a controlled release layer thereon.

In another embodiment there is provided a pharmaceutical composition wherein the release rate of drug from the pellets part of the pharmaceutical composition comprising a tabletted or encapsulated composition of a multitude of pellets is controlled by the amount or the percentage of the initial core/spheres of the pellets. Preferably, the amount of initial core is from about 15% to about 35% by weight of the controlled release coated pellets before tableting or capsule filling, such as from 20-30%.

In another embodiment the inert core is strengthened by applying a sub-coat on the initial core/sphere. In pharmaceutical compositions wherein pellets comprising the drug are compressed into tablets, the drug pellets are mixed with powder excipients to form a tableting blend. However, the size of the drug coated pellets, often larger than the particle size of the powder excipients, can cause a lack of uniformity of the tableting blend. The preferred uniformity of the tableting blend is such that the average assay of samples of the tableting blend each weighing the equivalent of one tablet lies within the range of 90 to 110 percent of the label dose and the relative standard deviation of the individual assays is less than or equal to 5 percent. The size of the drug pellets is therefore preferably small. When layering a large amount of drug on a small initial core a high degree of stress is exerted on the initial core. This stress may cause attrition particularly when the inert core comprises sugar spheres. To provide a higher degree of physical strength of the inert core without changing the dissolution rate of drug coated pellets, a sub-coat may be applied on an initial core/sphere. Preferably, the amount of the sub-coat is from about 10% to about 40% of the total weight of the sub-coated inert core, more preferably the amount of sub-coat is from about 15% to about 30% of the total weight of the sub-coated inert core, most preferably the amount of sub-coat is about 16% to about 20% of the total weight of the sub-coated inert core.

The inert core of each of the pellets in the pharmaceutical composition may comprise from about 50% to about 100% (per weight) of soluble substance. Preferably the inert core comprises from about 70% to about 90% (per weight) of soluble substances. A preferred initial core comprises a sugar sphere. Sugar spheres have been used in the pharmaceutical industry as excipients. Such sugar spheres used in pharmaceutical compositions generally contain not more than 92% of sucrose, calculated on the dried basis, the remainder consisting of maize starch. Commonly sugar spheres with a core size larger than 500 μm are used. The core size of the inert cores, preferably a sugar sphere, is between about 50 μm and about 500 μm, preferably between about 100 μm and about 400 μm, more preferably from about 250 μm to about 350 μm.

The inert core may comprise an initial core/sphere that is sub-coated with a layer of a plasticized film coating polymer. This sub-coating of an initial core/sphere provides physical strength to the inert core. The film coating polymer may be a hydrophobic or a hydrophilic polymer, or a combination of the two. Suitable film coating polymers can be cellulose derivative polymers or polymethacrylate polymers. Further, hydrophobic polymers or hydrophilic plasticizers, or a combination of several plasticizers can be used to plasticize the film coating polymers. These compounds of the polymeric sub-coat are mixed with solvents prior to their application onto the initial core/sphere. Suitable solvents for use in mixing the polymeric sub-coating compounds are selected from ethanol, isopropyl alcohol, acetone and purified water. For example a mixture of ethanol, acetone and water is preferred for use in mixing a mixture of the preferred sub-coating compounds EthylCellulose (as a film coating polymer), and plasticizers Dibyutyl Sebacate and Polyethylene Glycol (EC, DBS and PEG).

Preferably, the initial core/sphere is a sugar sphere which is sub coated with a mixture of polymers such as cellulose derivatives e.g. ethylcellulose and triethyl citrate, polyethylene glycol, dibutyl sebacate, and dibutyl phthalate, and wherein the sub-coating layer on the initial core/sphere does not alter the release rate of the drug for the pharmaceutical composition. A preferred sub-coat on the sugar spheres comprises ethyl cellulose as a hydrophobic film coating polymer and a combination of two or more plasticizers, at least one hydrophilic and at least one hydrophobic plasticizer. Suitable plasticizers may include for example polyethylene glycols, citrate esters, dibutyl sebacate, diethyl phthalate, and triacetin. Preferred plasticizers are polyethylene glycol and dibutyl sebacate as the hydrophilic and hydrophobic plasticizers respectively. Preferably, the sub-coat comprises about 75% to about 85% ethyl cellulose, about 10% to about 20% polyethylene glycol and about 3% to about 7% dibutyl sebacate by weight of the sub-coat. More preferably, the sub-coat comprises 80% ethyl cellulose, 15% polyethylene glycol and 5% dibutyl sebacate by weight of the sub-coat.

Alternatively the core may be an insoluble core onto which the active ingredient has been deposited for example by spraying. It may be made from silicon dioxide, glass or plastic resin particles. Suitable types of plastic material are pharmaceutically acceptable plastics such as polypropylene or polyethylene preferably polypropylene. Such insoluble cores may have a diameter in the range of 0.01-2 mm, preferably in the range of 0.05-1.0 mm and more preferably in the range of 0.1-0.7 mm.

Beta Blockers for Extended Release

In one embodiment, a beta blocker, such as Metoprolol or its acceptable pharmaceutical salt, may be applied on the inert core. No use of “Class 2” solvents (as defined by the FDA) is required to apply the active pharmaceutical ingredient (API), drug, onto the inert core forming a drug coated pellet. The FDA defines “Class 2” solvents as having inherent toxicity. The active ingredient is dispersed in water, preferably together with an acceptable binder excipient such as, but not limited to, polyvinyl pyrrolidone, cellulose derivatives polymers, or starch.

The beta blocker, such as metoprolol may be applied as a dispersion rather than a solution. Therefore it is preferred that the drug substance has physical properties that will allow a high yield in preparing drug coated pellets. Therefore, the drug substance preferably has a particle size distribution such that the d(0.9) value is less than about 80 μm. Preferably, the d(0.9) value for the particle size distribution of the drug substance is less than about 50 μm, more preferably less than about 30 μm. As a result, a concentrated dispersion for application can be produced which may shorten the production time.

The drug coated pellets may comprise from about 40% to about 90% (per weight) of the drug layer, preferably from about 50% to about 80% (per weight), more preferably from about 55% to about 75% (per weight).

Other beta blockers, such as Carvedilol or salts thereof, may be applied in a similar as indicated for Metoprolol.

Controlled Release Layer

The last layer applied on the pellets is a layer which controls the release of the active pharmaceutical ingredient. Pellets that have been coated with a controlled release layer may have a size between about 200 μm and about 800 μm. Preferably, the controlled release layer coated pellets have a size ranging from about 300 μm to about 700 μm, more preferably from about 400 μm to about 600 μm. In addition, the controlled release layer may comprise water soluble and insoluble components. Such components may be film forming polymers and plasticizers. For example, a film comprising a polymeric layer may be applied onto the drug coated pellets.

In the following three different types of extended release coatings are described.

First Extended Release Coating

In one embodiment the extended release film coat comprises i) an acrylic polymer ii) a surfactant and iii) sodium stearyl fumarate, wherein the film coat has been deposited from a water containing liquid.

Typically a film coating composition comprises

a) 25 to 35% by weight of an acrylic polymer dispersion

b) 0.1 to 4% by weight of a surfactant

c) 0.1 to 4% sodium stearyl fumarate and

d) a water-containing liquid to 100%.

In one embodiment there is provided film coatings which are suitable for giving extended release. Suitably the acrylic polymer used in this case comprises homogeneous particles wherein the polymer or copolymer has T_{g}<room temperature in aqueous dispersion but has T_{g}>room temperature in the dry state. Suitable polymers comprise acrylic acid and esters thereof particularly the methyl, ethyl, propyl and butyl esters; and methacrylic acid and esters thereof particularly the methyl, ethyl, propyl and butyl esters. Particularly preferred polymers are those provided under the tradenames Eudragit L30D® (Rohm Pharma) or Eudragit FS30D® (Rohm Pharma). Optionally further anti-tacking agents may be required.

Suitably the amount of the acrylic polymer in the film coating composition is in the range of 15 to 50% by weight. Preferably the amount of the acrylic polymer in the film coating composition is in the range of 20 to 40% by weight. More preferably the amount of the acrylic polymer in the film coating composition is in the range of 25 to 35% by weight.

Suitably the surfactant is one of the following: a nonionic surfactant, like sorbitan esters (Span series); polysorbates (Tween series); polyoxyethylated glycol monoethers (like the Brij series); polyoxyethylated alkyl phenols (like the Triton series or the Igepal series); alkyl glucosides (e g dodecylmaltoside); sugar fatty acid esters (e g sucrose laurate); saponins; etc: or mixtures thereof; ampholytic surfactants, like betaines; anionic surfactants, like sulphated fatty alcohols eg sodium dodecylsulphate SDS; sulphated polyoxyethylated alcohols; others like dioctyl sulphosuccinate; bile salts (e g dihydroxy bile salts like sodium deoxycholate, trihydroxy bile salts like sodium glycocholate, etc); fusidates (e g sodium dihydrofusidate); etc cationic surfactants, like ammonium compounds; soaps, fatty acids, and lipids and their salts, like alkanoic acids; (e g octanoic acid, oleic acid); monoglycerides (eg monolein), phospholipids which are neutral or positively or negatively charged (eg dialkyl phosphatidylcholine, dialkyl phosphatidylserine, etc); etc; more preferably the surfactant is a nonionic surfactant. Most preferably the surfactant is nonoxynol 100.

Suitably the amount of the surfactant in the film coating composition is in the range of 0.05 to 8% by weight. Preferably the amount of the surfactant in the film coating composition is in the range of 0.1 to 6% by weight. More preferably the amount of the surfactant in the film coating composition is in the range of 0.5 to 4% by weight.

In a most preferred embodiment the acrylic polymer and the surfactant are provided by Eudragit® NE30D in compositions, a film coats or formulations defined previously.

Suitably the amount of the sodium stearyl fumarate in the film coating composition is in the range of 0.05 to 8% by weight. Preferably the amount of sodium stearyl fumarate in the film coating composition is in the range of 0.1 to 6% by weight. More preferably the amount of sodium stearyl fumarate in the film coating composition is in the range of 0.5 to 4% by weight.

Suitably the water-containing liquid comprises water and a water miscible organic liquid for example lower alkanols e.g. ethanol, propanol or isopropanol. From a safety point of view is preferred that the proportion of the organic is kept to a minimum but small amounts are tolerable for example in the range of 0 to 20% by volume. Preferably the liquid is water.

The film-coating composition is particularly suitable for use as an aqueous film-coating composition wherein the film-coat is applied using water as the liquid. When the liquid is water the latex is preferably a poly(ethylacrylate-co-methylmethacrylate) copolymer, for example Eudragit NE30D® (Rohm Pharma). This process is particularly advantageous as it negates the need to use environmentally unacceptable organic solvents, some of which also present processing problems due to their inflammablility, while also eliminating many of the problems experienced with aqueous coatings described above.

Second Extended Release Coating

Alternatively, the film may comprise at least one film coating polymer and can be plasticized with one or more plasticizers. These plasticizers may differ from each other in their degree of solubility (hydrophobicity/hydrophilicity). By changing the ratio between the plasticizers and the film coating polymer, or the ratio between the different plasticizers (if more than one is used), one can control the rate of the release of the drug from the pellets. The controlled release layer of the beta blocker ER may comprise a hydrophobic film coating polymer such as for example ethylcellulose and a combination of at least two plasticizers, at least one hydrophilic and one hydrophobic plasticizer, for example polyethylene glycol and dibutyl sebacate. Preferably, the ratio of hydrophobic to hydrophilic plasticizer in the controlled release layer of the pharmaceutical composition is from 3:1 to 1:3, more preferably the ratio is 1:1.

Furthermore, the controlled release layer may comprise at least about 70% water insoluble compounds (per weight of the controlled release layer). Preferably, the controlled release layer comprises at least about 80% and more preferably at least about 90% water insoluble compounds (per weight of the controlled release layer). Suitable water insoluble compounds are for example cellulose derived polymers. These controlled release layer compounds are mixed with solvents prior to their application onto the drug coated pellets. Suitable solvents for use in mixing the controlled release layer compounds are selected from ethanol, isopropyl alcohol, acetone and purified water. A mixture of ethanol, acetone and water is preferred for use in mixing the controlled release layer compounds especially where the controlled release layer compounds are a mixture of ethylcellulose, dibutyl sebacate and polyethylene glycol.

The method of preparing the beta blocker ER component may comprise sub-coating an initial core/sphere forming an inert core. Sub-coating an initial core/sphere comprises mixing a film coating polymer with one or more plasticizers in a solvent forming a coating mixture. Such mixture may be a solution, suspension or slurry for applying a coating layer on a surface. The coating mixture is applied to the initial core/sphere forming a sub-coated initial core/sphere which is used as an inert core. The film coating polymer may be a hydrophobic or a hydrophilic polymer, or a combination of the two. Suitable film coating polymers can be cellulose derivative polymers or polymethacrylate polymers, preferably ethylcellulose. The amount of ethylcellulose is preferably from about 75% to about 85% more preferably about 80% of the total amount of the weight of the sub-coat. Further, hydrophobic polymers or hydrophilic plasticizers, or a combination of several plasticizers can be used to plasticize the film coating polymers. These compounds of the polymeric sub-coat are mixed with solvents prior to their application onto the initial core/sphere. Suitable solvents for use in mixing the polymeric sub-coating compounds are selected from ethanol, isopropyl alcohol, acetone and purified water. A mixture of ethanol, acetone and water is preferred for use in mixing the polymeric sub-coating compounds.

Suitable plasticizers for use in sub-coating an initial core/sphere are selected from polyethylene glycol, dibutyl sebacate, and dibutyl phthalate. Preferred plasticizers are polyethylene glycol and dibutyl sebacate as the hydrophilic and hydrophobic plasticizers respectively. Preferred amounts of plasticizers used in the method are about 10% to about 20% polyethylene glycol and 3% to about 7% dibutyl sebacate by weight of the sub-coat. More preferably, about 15% polyethylene glycol and 5% dibutyl sebacate as plasticizer.

For the extended release coat, the amount of ethylcellulose is preferably from about 75% to about 85% more preferably about 80% of the total amount of the weight of the coat. Suitable plasticizers for use in the ER-coating are selected from polyethylene glycol, dibutyl sebacate, and dibutyl phthalate. Preferred plasticizers are polyethylene glycol and dibutyl sebacate as the hydrophilic and hydrophobic plasticizers respectively. Preferred amounts of plasticizers used in the method are about 5% to about 20% polyethylene glycol and dibutyl sebacate by weight of the ER-coat. More preferably, about 10% polyethylene glycol and 10% dibutyl sebacate as plasticizer.

In one embodiment, a metoprolol ER tablet comprises components according to table 4.

TABLE 4
Metoprolol ER tablet.

| | | |
|---|---|---|
| | | Percent total |
| Material | Weight | pellet weight |

Sub-coated pellets

| | | |
|---|---|---|
| Sugar spheres (250-355 μm) | 598.00 | 22.3 |
| Ethyl cellulose 7 cps | 92.00 | 3.4 |
| Polyethylene glycol 400 | 17.25 | 0.6 |
| Dibutyl sebacate | 5.75 | 0.2 |

Drug layer

| | | |
|---|---|---|
| Metoprolol succinate | 1092.50 | 40.9 |
| Polyvinyl pyrrolidone | 276 | 10.3 |
| povidone (PVP K-30) | | |

Controlled release film layer

| | | |
|---|---|---|
| Ethyl cellulose 100 cps | 473.8 | 17.7 |
| Polyethylene glycol 400 | 59.23 | 2.2 |
| Dibutyl sebacate | 59.23 | 2.2 |

In a preferred method of preparing the beta blocker ER part of the composition, the method comprises the following steps; a) providing sugar spheres as initial cores; b) coating the sugar spheres with a sub-coat comprising mixing a film of a hydrophobic polymer, a soluble (hydrophilic) plasticizer, and an insoluble (hydrophobic) plasticizer with a solvent mixture of e.g. acetone, ethanol 95%, and water and spraying the mixture onto the sugar spheres to create a sub-coat on the sugar spheres resulting in an inert core; c) coating the sub-coated sugar spheres (inert cores) with a drug layer comprising mixing the drug, such as metoprolol succinate, and a binder, preferably povidone (PVP K-30) with preferably water, forming an aqueous dispersion and applying the dispersion onto the sub-coated pellets (inert cores) forming drug coated pellets; d) applying a third layer on the drug coated pellets comprising dissolving a hydrophobic film coating polymer, an hydrophilic plasticizer and an hydrophobic plasticizer in a solvent mixture of e.g. acetone, ethanol 95%, and water forming a mixture and spraying the mixture onto the drug coated pellets to create controlled release drug coated pellets; e) mixing the controlled release drug coated pellets with a powder mixture of one or more excipients forming a final blend; f) compressing the final blend into tablets or filling the final blend into capsules; and g) optionally film coating the tablets for cosmetic purposes.

In this method the hydrophobic polymer is preferably ethyl cellulose (EC), the soluble/hydrophilic plasticizer is preferably polyethylene glycol (PEG), and the insoluble/hydrophobic plasticizer is preferably dibutyl sebacate (DBS). Further, in preparing a mixture for coating the sugar spheres with a sub-coat, and the drug coated pellets with a controlled release layer, ethyl cellulose is preferably first dissolved in acetone and ethanol 95%, then PEG and DBS are added, followed by adding water and mixing the solution till it is homogenized. Preferably, the spraying of a solution or dispersion onto sugar spheres or drug coated pellets in the method uses a fluidized bed coater with a Wurster insertion. Furthermore, the binder, used in coating the sub-coated sugar spheres with a drug layer, facilitates binding of the drug to the inert core of sub-coated sugar spheres. Moreover, in this method the ratio of powder mixture to controlled release drug coated pellets in the final tableting blend is preferably from about 20% to about 60% (by weight), more preferably from about 30% to about 50% (by weight), most preferably from about 35% to about 45% (by weight). As a result a uniform final tableting blend and tablets are produced.

Third Extended Release Coating

An extended release phase may comprise at least one high viscosity hypromellose (HPMC) ingredient. HPMC is a water soluble matrix-forming polymer used to provide an extended release effect of metoprolol. The viscosity of the HPMC used in the ER phase may be up to 100.000 centipoise such as in the range of about 3500-6000 cps.

An extended release layer with a therapeutically effective amount of a beta blocker, such as metoprolol or carvediol, can be made with high viscosity hypromellose alone.

In other embodiments, the extended release layer comprises a therapeutically effective amount of a beta blocker, such as metoprolol or carvediol, at least one high viscosity hypromellose, at least one binding agent, a low viscosity hypromellose, at least one modified starch, and optionally one or more other pharmaceutically acceptable intragranular components including but not limited to a second pharmaceutically acceptable active ingredient, other pharmaceutically acceptable excipients and/or adjuvants. In one embodiment, the ratio of high-viscosity hypromellose to low viscosity hypromellose is about 3.3 to about 0.85. In another embodiment the ratio of high to low is about 3:1.

Suitably, the viscosity of the low viscosity hypromellose is in the range of about 10-30 centipoises. In another embodiment the low viscosity is about 15 centipoises.

The amount of at least one binding agent in the extended release phase of a bilayer tablet may be from about 0.5% to about 3% w/w. In one embodiment there are at least two binding agents present in the ER phase. Suitably the amount of at least one modified starch in the extended release phase of the bilayer tablet is from about 0.5% to about 3% w/w. In one embodiment, the amount of modified starch is about 1% w/w of the ER phase. In one embodiment there are at least two modified starches present in the ER phase. Suitably, the modified starch is pre-gelatinized.

Suitably, the amount of the high viscosity hypromellose present in the extended release phase is from about 3%>to about 7%>of the extended release phase formulation weight. In another embodiment, the amount of high viscosity hypromellose is from about 4% to about 6%. In still other embodiments, an amount of >20% hypromellose is used in the extended release phase.

In yet another embodiment the amount of high viscosity HPMC is present in an amount of about 5% w/w extended release phase formulation weight.

Suitably, the amount of the low viscosity hypromellose present in the extended release phase is from about 0.5% to about 3% of the extended release phase formulation weight. In another embodiment, the amount of low viscosity hypromellose is from about 1% to about 2% of the extended release phase formulation weight.

Alternatively, the total amount of cellulosic derivatives of HPMC present in the ER granulate range from about 3% to about 10% by weight of the total amount of extended release components. This encompasses both the high and the low viscosity HPMC's.

In one embodiment the ER phase comprises metoprolol, povidone, pre-gelatinized corn starch, and a high and low viscosity HPMC.

In one embodiment the ER phase comprises carvedilol, povidone, pre-gelatinized corn starch, and a high and low viscosity HPMC.

Tablets and Capsules

The film coated beads or spheres may be provided in sachets or formulated as a capsule, for example a hard gelatin capsule, or compressed to form tablets using known methods with the optional addition of other pharmaceutically acceptable additives and with the addition of the beta blocker IR and tesofensine components herein described. Coated beads to be compressed into a tablet are obtained by conventional techniques known to those skilled in the art.

Also, during this process suitable other agents can be added. For example, during the tabletting step suitable fillers, eg microcrystalline cellulose, lactose monohydrate, talc. sodium stearyl fumarate etc can be utilised to give acceptable compression characteristics of the formulation, e g hardness of the tablet.

These additives can be granulated in one of the conventional granulation methods. However, preferably there is provided a set of additives, for example a powder mixture that can be directly compressed into tablets. Such powder mixture serves as a filler, cushioning, disintegrant, glidant, and lubricant mixture. Furthermore, the ratio of controlled release drug coated pellets to additives in the final (e.g. tableting) blend of the present pharmaceutical composition is of particular importance to prepare a uniform product e.g. tablets.

To prepare a uniform product, preferably at least 50% (by weight) of the powder mixture may have particle sizes between about 30 μm to about 800 μm, preferably from about 80 μm to about 600 μm, more preferably from about 100 μm to about 300 μm. More preferably, at least 65% (by weight) of the powder mixture has particle sizes between about 30 μm to about 800 μm, preferably from about 80 μm to about 600 μm, more preferably from about 100 μm to about 300 μm. Most preferably, at least 80% (by weight) of the powder mixture has particle sizes between about 30 μm to about 800 μm, preferably from about 80 μm to about 600 μm, most preferably from about 100 μm to about 300 μm.

Furthermore, the amount of controlled release drug coated pellets in the final tableting blend is preferably from about 20% to about 60% (by weight) in order to prepare such uniform product. More preferably, the amount of controlled release drug coated pellet in the final tableting blend is from about 30% to about 50% (by weight), most preferably from about 35% to about 45% (by weight).

Suitable powder mixtures comprise, but are not limited to, mixtures of two or more of the following compounds; Starlac(R) (a spray-dried compound consisting of 85% alpha-lactose monohydrate and 15% maize starch dry matter available from Meggle), Cellactose(R) (a spray-dried compound consisting of 75% alpha-lactose monohydrate and 25% cellulose powder dry matter available from Meggle), Parteck(R) (A Directly Compressible Sorbitol available from Merck KGaA), Crospovidone, Silicon Dioxide, Magnesium Stearate, Talc, Zinc Stearate, Polyoxyethylene Stearate, Stearic Acid, sodium stearyl fumarate Cellulose derivatives, microcrystalline cellulose and lactose monohydrate.

If the dosage form is a bi- or tri-layer tablet, the immediate release layer(s) may be compressed directly on a previously partly compressed extended release layer, or alternatively, the extended release layer may be compressed onto previously partly compressed immediate release layer(s).

The compositions can be formulated by conventional methods of admixture such as granulating, blending, filling and compressing. For example, tablets can be produced by a wet granulation process, where the immediate release phase and extended release phase are separately prepared. Suitably, for either the immediate release or extended release phase, the active drug substance and excipients are screened and mixed in a high shear mixer granulator or fluid bed dryer. The blend is granulated by the addition of a granulating solution (typically purified water, disintegration agent dissolved/dispersed in purified water, or drug dissolved/dispersed in purified water or a suitable solvent) sprayed into the high shear mixer granulator or fluid bed dryer. If desired wetting agents e.g., surfactants can be added. The resulting granules (optionally pelletized) are dried usually with residual moisture of 1-5% by tray, fluid bed or microwave drying techniques. The dried granules are milled to produce a uniform particle size, the granules are blended with extragranular excipients as necessary, typically a lubricant and glidant (e.g., magnesium stearate, silicon dioxide). The separately prepared immediate release and extended release granules can then be compressed together using a rotary tablet press (such as a bilayer tablet press) if desired. If the dosage form is a single layer tablet, then the extended release granules are admixed with the immediate release extragranular components and compressed together using a rotary tablet press, etc. These resulting tablets can all be coated in a pan coater typically with a 1-5% aqueous film coat, followed by a wax polishing.

Alternatively tablets can be produced by a direct compression process. Suitably the active drug substance and excipients for the immediate release and extended release phases are separately screened and mixed in a suitable blender e.g., a cone, cube or V-blender. Other excipients are added as necessary, and further blended. The separately prepared immediate release and extended release phases can be combined and compressed together using a rotary tablet press as hereinbefore described. The resulting tablets can be coated in a pan coater.

Tablets can also be prepared by using both methods of wet granulation and direct compression. For example the extended release phase can be prepared by wet granulation as described herein, while the immediate release phase can be prepared by blending the excipients for direct compression. The two phases can then be combined and compressed together as hereinbefore described.

Immediate Release Phase(s)

The immediate release phase(s) may be prepared by combining a directly compressible commercially available grade of the beta blocker, such as metoprolol, and tesofensine with a lubricant, and one or more disintegrating agents if necessary or desired. Binders and other excipients and/or adjuvants may be included in the immediate release layer(s), also if necessary or desired. The beta blocker and tesofensine in the immediate release layer may be combined with a modified starch such as a pre-gelatinized starch, e.g., corn starch, polyethylene glycol, and a disintegrant, or super disintegrant such as croscarmellose sodium or Explotab®, a binder such as methylcellulose or hypromellose polymer, plasticizer, pigment and a lubricant.

The immediate release phases may comprise two different layers of the beta blocker and tesofensine, respectively. Alternatively, the immediate release phases may be combined into one and the same layer. The immediate release phases may also be formulated into an extragranular phase of a tablet or be granulated into one or two different immediate release granules. For tesofensine, the preferred formulation is a granulation of tesofensine compared to direct compression of tesofensine as the dose is relatively low.

Monolith Dosage Form

In one embodiment, there is only a single layer tablet having an extended release intra-granular phase and two immediate release extra-granular phases. The extended release phase will be comprised of an intra-granular component of the beta blocker and excipients as described above. These components form the ER granulate. The ER blend could be made into pellets and compressed accordingly with the extra-granular immediate release blend.

A suitable extra-granular component or phase, i.e., the immediate release phases, may be prepared by combining a directly compressible commercially available grade of a beta blocker, such as metoprolol, and tesofensine citrate with a lubricant, and one or more disintegrating agents if necessary or desired. As mentioned above for tesofensine the preferred process is to prepare a granulate of tesofensine before compression. Binders and other excipients and/or adjuvants may be included in the extra-granular phase if necessary or desired. Alternatively, an extra-granular component can be prepared by combining the beta blocker, such as metoprolol, and tesofensine with a modified starch, such as a pre-gelatinized starch, e.g., corn starch, a disintegrant or super disintegrant, such as croscarmellose sodium, a binder and a lubricant.

Excipients

The present compositions may include components that functions as a binder or binding agent. Suitably, the binding agent may comprise a first binding agent and a second binding agent. Suitable binding agents for use herein include conventional binding agents used in the art such as gelatin, starches, povidone, polymers and cellulose derivatives or combinations thereof.

Suitably, the starch, is of vegetable origin, such as corn (or maize) starch, modified corn starch, wheat starch, modified wheat starch, potato starch, or pre-gelatinized starch e.g., available commercially as Starch 1500 G or Prejel; or a combination of two or more thereof.

If the binding agent includes a cellulosic derivative such as hydroxypropyl cellulose (HPC) (of low to medium viscosity) e.g., as may be available commercially under the brand name Klucel® from the Aqualon division of Hercules Inc., Dow Chemical Company e.g., Klucel G F, Klucel J F, Klucel L F and Klucel E F; microcrystalline cellulose (MCC), carboxymethylcellulose (MC), sodium carboxymethylethyl cellulose; or a combination of two or more thereof. Combinations of a cellulosic derivative with other binding agents noted above are also envisaged. Generally the total amount of cellulosic derivatives present in the granulate are in an amount ranging from about 3% to about 10% by weight of the extended release components. It is recognized in the art that certain cellulosic derivatives, such as hypromellose, will have varying roles in a formulation, depending upon the amount used. For example hypromellose (low or medium viscosity) may function as a binding agent, a coating agent, or as a matrix forming agent.

While a binding agent is present as an intra-granular component, it is recognized that a modest amount of binding agent e.g., up to about an additional 3.0%>−10.0% by weight of the intra-granular binding agent content of the composition, may also be present extra-granularly.

In one embodiment, suitably the starch is pre-gelatinized starch. Pre-gelatinized starch is a starch that has been chemically and/or mechanically processed. Typically pre-gelatinized starch contains 5% of free amylase, 15% of free amylopectin, and 80% unmodified starch. Pre-gelatinized starch may be obtained from corn (or maize), potato or rice starch.

The granulate provides an intimate admixture of a combination of ingredients and may then be mixed with one or more pharmaceutically acceptable extra-granular components of the composition i.e., with any pharmaceutically acceptable ingredient e.g., a diluent, flavor, sweetening agent, binder, disintegrant, glidant, lubricant, anti-adherent, anti-static agent, anti-oxidant, desiccant, or a second pharmaceutically acceptable active agent. It is recognized that these same ingredients may be present both as an intra-granular and as an extra-granular ingredient.

As noted above there are other inactive ingredients that may optionally be employed in relatively small quantities, which include lubricants, flow agents, and binders that facilitate compression.

Suitable disintegrating agents include a non-super disintegrant, a super disintegrant or a combination of both. Suitable non-super disintegrants include conventional disintegrants such as starch (corn or maize), pre-gelatinized starch e.g., Starch 1500 G, clays (e.g. VEEGUM (Vanderbilt Minerals, LLC) or Bentonite (an absorbent aluminium phyllosilicate clay consisting mostly of montmorillonite)), microcrystalline cellulose, cellulose or powdered cellulose. It is recognized in the art, that some excipients may perform more than one role in a given pharmaceutical formulation. For example certain excipients, e.g., starches including pre-gelatinized starch, and microcrystalline cellulose (hereinbefore identified as binding agents) function as both binders and disintegrants.

A “super disintegrant” represents a class of disintegrating agent which may generally be used in lower amounts in pharmaceutical preparations, as compared to conventional disintegrants. Examples of super disintegrants include sodium starch glycolate, the sodium salt of carboxymethyl starch, modified cellulose and cross-linked polyvinyl pyrrolidone. Sodium starch glycolate is available commercially under the trade names Explotab® (Edward Mendell Co. JRS Pharma), Primojel® (Generichem Corp; DFE Pharma) and Tablo® (Blanver, Brazil). An example of modified cellulose includes croscarmellose sodium, the sodium salt of carboxymethyl cellulose. Croscarmellose sodium is available commercially under the trade names AcDiSol® (FMC Corp.), Nymcel ZSX® (Nyma, Netherlands), Primellose® (Avebe, Netherlands), Solutab® (Blanver, Brazil). An example of a cross-linked polyvinyl pyrrolidone includes crospovidone, and is commercially available under the trade names Kollidon CL® or Kollidon CL-M (Basf Corp.), and Polyplasdone XL® (ISP Corp; Ashland). A suitable super disintegrants includes croscarmellose sodium or sodium starch glycolate (e.g. Explotab® (JRS Pharma)) or a combination thereof. A super disintegrant may be used extragranularly, in an amount ranging from about 0.5% to about 5.0% by weight of the composition. Suitable preservative or antimicrobial agents for use include potassium sorbate or a paraben, i.e., one or more hydroxy benzoic acid esters e.g., methyl, ethyl, propyl or butyl, suitably singularly or as mixtures. Parabens are commercially available under the Nipa® brand name, e.g., Nipasept® sodium (Aako BV).

Suitable lubricants include magnesium, calcium or sodium stearate, stearic acid or talc that may be added in suitable amounts. In one embodiment the lubricant is magnesium stearate.

Suitable flow agents include silicon dioxide (e.g. Cab-O-Sil® (Cabot Corporation), Syloid™ (W.R. Grace & Co.)) and colloidal silicon dioxide (Aerosil® (Evonik Resource Efficiency GmbH)), that may be added in an amount from about 0.5% to about 1% by weight.

The compressed tablet may further comprise a film coat e.g., hypromellose or polyvinyl alcohol-part.hydrolised (PVA). Suitably the film coat is a transparent film coat e.g., a dye, although an opaque film coat e.g., as obtained when using a film coat in combination with an opacifier or a pigment such as titanium dioxide or a lake may also be used. For example one commercially available film coat is an Opadry® coating system from Colorcon.

Medical Use of Pharmaceutical Composition

In one aspect, the present invention relates to the pharmaceutical composition as defined herein for use as a medicament.

In one embodiment, the pharmaceutical composition as defined herein is for use in the treatment of a disorder or condition selected from the group consisting of obesity, an obesity associated disorder, Prader-Willi syndrome, hypothalamic obesity, hyperphagia, metabolic syndrome, dyslipidemia, atherosclerosis, drug-induced obesity, overeating disorders, bulimia nervosa, binge eating disorder, compulsive over-eating, impaired appetite regulation, nonalcoholic fatty liver disease (NAFLD) and nonalcoholic steatohepatitis (NASH).

Obesity is defined herein as a medical condition in which excess body fat has accumulated to the extent that it may have an adverse effect on health, leading to reduced life expectancy and/or increased health problems in general. Thus, in one embodiment the subject to be treated with the composition of the present disclosure is obese.

Body mass index (BMI) is a measure which compares weight and height. People are generally considered overweight or pre-obese if the BMI is between 25 and 30 and obese if the BMI is over 30. Morbidly obese subjects have a BMI over 35. In one embodiment the subject has a BMI above 25 kg/m², such as above 30 kg/m², for example above 35 kg/m², such as above 40 kg/m². In one embodiment, the subject has a BMI above 30 kg/m². In one embodiment, the subject has a BMI above 35 kg/m².

In one embodiment, the composition of the present disclosure is for use in the treatment of an obesity associated disorder, such as a disease or disorder selected from the group consisting of diabetes, metabolic syndrome, dyslipidemia, atherosclerosis, drug-induced obesity, overeating disorders, bulimia nervosa, binge eating disorder, compulsive over-eating, impaired appetite regulation, nonalcoholic fatty liver disease (NAFLD) and nonalcoholic steatohepatitis (NASH).

In one embodiment, the composition of the present disclosure is for use in the treatment of diabetes, such as type 1 diabetes, type 2 diabetes, prediabetes and gestational diabetes. Preferably, the diabetic subject is obese.

In one embodiment, the composition as described herein leads to an alleviation or improvement of diabetic complications.

Type 1 diabetes (diabetes mellitus type 1) is a form of diabetes that results from the autoimmune destruction of the insulin-producing beta cells in the pancreas. In type 1 diabetes, hypertension may reflect the onset of diabetic nephropathy.

Type 2 diabetes is a metabolic disorder that is characterized by hyperglycemia in the context of insulin resistance and a relative lack of insulin. Type 2 diabetes makes up about 90% of cases of diabetes, with the other 10% due primarily to diabetes mellitus type 1 and gestational diabetes. Obesity is thought to be the primary cause of type 2 diabetes in people who are genetically predisposed to the disease.

Pre-diabetes is used interchangeably herein with intermediate hyperglycaemia. Intermediate hyperglycaemia is a biochemical state in which a person has glucose levels above the normal range, but does not yet meet the criteria for a diagnosis of diabetes. The primary aim of management of intermediate hyperglycaemia is to prevent progression to diabetes.

A pre-diabetic subject may have one or more of impaired fasting glycaemia (IFG) and/or impaired glucose tolerance (IGT) and/or elevated glycated haemoglobin (HbA_{1c}) levels.

Weight loss can prevent progression of pre-diabetes into diabetes and can also markedly improve clinical symptoms of type 2 diabetes. Thus, weight loss is an attractive treatment strategy for pre-diabetic subjects and subjects suffering from type 2 diabetes.

In one embodiment the subject is an obese, pre-diabetic human. In one embodiment the subject is an obese subject suffering from type 2 diabetes.

Gestational diabetes is a condition in which women without previously diagnosed diabetes exhibit high blood glucose levels during pregnancy (especially during their third trimester). Gestational diabetes is caused when insulin receptors do not function properly.

The WHO diabetes diagnostic criteria are shown in the table below.

| | 2 hour | Fasting | |
|---|---|---|---|
| | glucose* | glucose | HbA_{1c} |
| | mmol/1 | mmol/1 | mmol/mol |
| Condition | (mg/dl) | (mg/dl) | (DCCT %) |
| Normal | <7.8 (<140) | <6.1 (<110) | <42 (<6.0) |
| Impaired | <7.8 (<140) | ≥6.1(≥110) & | 42-46 (6.0-6.4) |
| fasting | | <7.0(<126) | |
| glycaemia | | | |
| Impaired | ≥7.8 (≥140) | <7.0 (<126) | 42-46 (6.0-6.4) |
| glucose | | | |
| tolerance | | | |
| Diabetes | ≥11.1 (≥200) | ≥7.0 (126) | ≥48 (≥6.5) |
| mellitus | | | |

*Venous plasma glucose 2 hours after ingestion of 75 g oral glucose load

The subject benefitting from treatment with the composition of the present disclosure may also be a subject suffering from an obesity-associated disorder or condition, such as one selected from the group consisting of diabetes, metabolic syndrome, dyslipidemia, atherosclerosis, drug-induced obesity, overeating disorders, bulimia nervosa, binge eating disorder, compulsive over-eating, impaired appetite regulation, nonalcoholic fatty liver disease (NAFLD) and nonalcoholic steatohepatitis (NASH).

In one embodiment, the composition of the present disclosure is for use in the treatment of metabolic syndrome, such as for the treatment of an obese subject suffering from metabolic syndrome.

In one embodiment the composition of the present disclosure is for use in the treatment of fatty liver disease, such as nonalcoholic fatty liver disease (NAFLD) or nonalcoholic steatohepatitis (NASH). The subject suffering from NAFLD or NASH is preferably obese.

In one embodiment, the composition of the present disclosure is for use in the treatment of nonalcoholic fatty liver disease (NAFLD).

In one embodiment, the composition of the present disclosure is for use in the treatment of nonalcoholic steatohepatitis (NASH).

Nonalcoholic fatty liver disease (NAFLD) is a cause of a fatty liver, occurring when fat is deposited in the liver (steatosis) due to other causes than excessive alcohol use. NAFLD is the most common liver disorder in Western industrialized nations. NAFLD is associated with insulin resistance and metabolic syndrome (obesity, combined hyperlipidemia, diabetes mellitus (type II) and high blood pressure). Non-alcoholic steatohepatitis (NASH) is the most extreme form of NAFLD, and is a major cause of cirrhosis of the liver. NASH is a state in which the steatosis is combined with inflammation and fibrosis (steatohepatitis).

In one embodiment, the composition of the present disclosure is for use in a method of decreasing liver fat and/or visceral adiposity. Reduction of liver fat and/or visceral adiposity has been shown to be effective in the treatment of fatty liver disorders. Tesofensine significantly decreases waist circumference and sagittal diameter (Astrup et al., 2008, Lancet 372: 1906-13); hence, tesofensine is capable of reducing visceral adiposity.

The composition of the present disclosure is preferably administered to a subject in need thereof once a day. However, in certain embodiments, the composition may be administered more than once a day, such as twice a day or alternatively less than once a day, such as once every second or third day depending on the specific formulation and concentration of the individual components of the composition. The subject treated is preferably a human, such as an adult human aged 18 or older.

In one embodiment, the present disclosure relates to use of the composition as disclosed herein in the manufacture of a medicament for the treatment of diabetes, obesity or an obesity associated disorder.

In one embodiment, the pharmaceutical composition as defined herein is for use in the treatment hyperphagia and/or for use in reduction or maintenance of body weight in Prader-Willi patients.

In one embodiment, the pharmaceutical composition is administered one, two or three times daily. In one embodiment, the beta blocker is administered one, two or three times daily. Preferably, the pharmaceutical composition is administered once daily.

### EXAMPLES

### Example 1. Phase 2a Trial Entitled “A Double-Blind, Randomized, Placebo-Controlled, Multiple-Dose, Multi-Centre Safety and Efficacy Study of Co-Administration of Tesofensine/Metoprolol in Subjects with Prader-Willi Syndrome (PWS) Step 1”

This exploratory Phase 2a trial comprised in of a total of nine adult patients with Prader-Willi syndrome (PWS) of which six patients received co-administration of tesofensine (0.50 mg)/metoprolol (50 mg) (A, active treatment—Tesomet) and three patients received placebo (P). The initial plan was to complete 10-20 adult subjects with PWS. However, ultimately nine patients were randomized as more subjects were not available at the two participating sites.

The main objective of this study was to examine the effect of co-administration of tesofensine/metoprolol on body weight in subjects with PWS. Further objectives were:
To establish a pharmacokinetic profile of tesofensine and metoprolol in subjects with PWS.
To examine the effect of co-administration of tesofensine/metoprolol on glycaemic control and lipid profile in subjects with PWS
To examine the effect of co-administration of tesofensine/metoprolol on HR and BP in subjects with PWS
To examine the effects of co-administration of tesofensine/metoprolol on body composition in subjects with PWS
To evaluate overall safety and tolerability of co-administration of the tesofensine/metoprolol in subjects with PWS

Subjects

Minimum of 10 and maximum of 15 randomized adult subjects and minimum of 10 and maximum of 15 randomized pediatric subjects with PWS, in total minimum of 20 and maximum of 30 randomized subjects.
Number of planned: 10-15 adults+10-15 children with PWS
Number of completed: 10-15 adult+10-15 children with PWS

TABLE 5
Number of patients per Visit.

| | | Visit 1 | Visit 2 | Visit 3 | Visit 4 | Visit 5 | Visit 6 | Visit 7 | Visit 8 |
|---|---|---|---|---|---|---|---|---|---|
| Site | Treatment | (day 0) | (day 7) | (day 14) | (day 21) | (day 28) | (day 35) | (day 42) | (day 49) |
| Site | Placebo | 1 | 1 | 1 | 0 | 0 | 0 | 0 | 0 |
| 01 | IMP | 3 | 3 | 3 | 3 | 3 | 2 | 2 | 2 |
| CZ | | | | | | | | | |
| Site | Placebo | 2 | 2 | 2 | 2 | 2 | 1 | 3 | 2 |
| 02 | IMP | 3 | 3 | 3 | 3 | 3 | 3 | 3 | 3 |
| HU | | | | | | | | | |
| Total | Placebo | 3 | 3 | 3 | 2 | 2 | 1 | 3 | 2 |
| | IMP | 6 | 6 | 6 | 6 | 6 | 5 | 5 | 5 |

| | | Visit 9 | Visit 10 | Visit 11 | Visit 12 | Visit 13 | Visit 14 | Visit 15 |
|---|---|---|---|---|---|---|---|---|
| | | (day | (day | (day | (day | (day | (day | (day |
| Site | Treatment | 56) | 63) | 70) | 77) | 84) | 91) | 105) |
| Site 01 | Placebo | 0 | 0 | 0 | 0 | 0 | 0 | 0 |
| CZ | IMP | 2 | 1 | 1 | 1 | 1 | 1 | 1 |
| Site 02 | Placebo | 2 | 2 | 2 | 2 | 2 | 2 | 2 |
| HU | IMP | 3 | 3 | 1 | 1 | 1 | 1 | 1 |
| Total | Placebo | 2 | 2 | 2 | 2 | 2 | 2 | 2 |
| | IMP | 5 | 4 | 2 | 2 | 2 | 2 | 2 |

Methodology

Two-centre, double-blind, placebo-controlled, randomized, and multiple-dose clinical study. Study medication will be administered for 91 days:
10-15 adult subjects with PWS will be treated.
Data Safety Monitoring Board (DSMB) review—following the completion of the treatment of the adult subjects, unblinded efficacy, safety, PK data as well as all data from the study in subjects with type 2 diabetes (TM001) will be reviewed by an independent DSMB. Following the DSMB's approval the study will proceed to:
Step 2-10-15 children with PWS, see Example 2.

Arm 1) tesofensine 0.50 mg+metoprolol 50 mg administered once a day, in the morning with a meal

or

Arm 2) Placebo tablets matching tesofensine+metoprolol administered once a day, in the morning with a meal

Each tablet will be formulated separately; a currently available commercial formulation of extended-release metoprolol will be used.

In the study a mean trough plasma concentration of ˜20 ng/mL was achieved with a dosing of 0.5 mg tesofensine which can also be seen in the below Table 6.

TABLE 6
Mean trough plasma concentration of tesofensine.

| TM002 study | V5(n = 6) | V9 (n = 5) | V14(n = 2) |
|---|---|---|---|
| Tesofensine | 18 ± 9 | 21 ± 8 | 23 ± 1 |
| [ng/mL] | | | |

Two patients had concentrations of 34.1, 34.2 ng/mL, respectively.

Unexpectedly, the patients treated with Tesomet in the Step 1 of the study were exposed to on average two to four times higher concentrations than anticipated from previous clinical trials.

The observed very high plasma concentration in this study may be explained in part by a lower metabolic rate and clearance of tesofensine in patients with PWS and also potentially by the extremely high body fat percentage in these patients.

Efficacy:

The clinical study achieved a positive outcome on the primary endpoint with a clinically meaningful reduction in weight for patients treated with Tesomet compared to placebo, see FIG. 1b. After 8 weeks the mean change in body weight was 5.00% (n=5) for patients receiving Tesomet compared to 0.46% (n=2) for patients receiving placebo. After 12 weeks the change in body weight was 6.76% (n=2) for patients receiving Tesomet compared to 0.75% (n=2) for patients receiving placebo. The average weight reduction was equal to 4.78 kg after 8 weeks and 7.95 kg after 12 weeks for patients receiving Tesomet, see FIG. 1a. There was a significant variance in weight loss between patients where two patients experienced a weight loss of up to 7 and 14 kg and one patient achieved a weight loss of 2 kg. There was a substantial reduction in waist circumference of 7.3 cm after 8 weeks and 10 cm after 12 weeks for patients treated with Tesomet. The variance was higher on this parameter and there was also a reduction in the placebo group with 4 cm and 6.5 cm for the two time points, respectively.

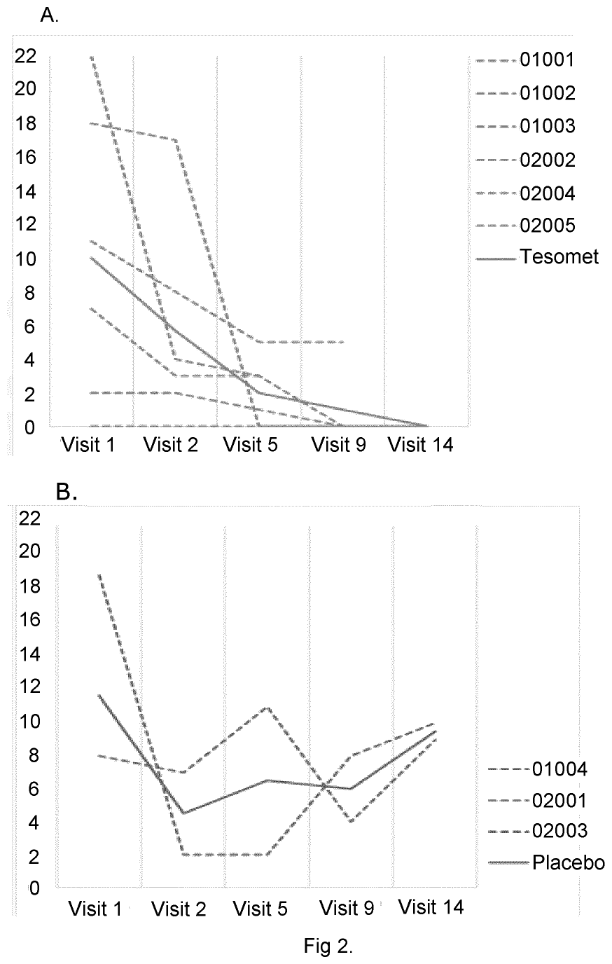

A significant reduction in the craving for food in patients treated with Tesomet was seen. The total score fell from 10 (n=6) at baseline to 1 (n=5) after 8 weeks and to 0 (n=2) after 14 weeks where 0 is equivalent to no observations of hyperphagia. After just one week (V2) of treatment the score fell from 10 at baseline to 5.67 (n=6) equivalent to a reduction of 43%. The observed hyperphagia score in the placebo group varied over time due to the low number of subjects (n=2), but it did not change substantially from baseline. The individual measurements over time can be seen in FIG. 2.

### Example 2. Phase 2a Trial Entitled “A Double-Blind, Randomized, Placebo-Controlled, Multiple-Dose, Multi-Centre Safety and Efficacy Study of Co-Administration of Tesofensine/Metoprolol in Subjects with Prader-Willi Syndrome (PWS) Step 2”

Arm 1) tesofensine 0.125 mg+metoprolol 25 mg administered once a day, in the morning with a meal-first, an initial titration dose of tesofensine 0.0625 mg+metoprolol 25 mg will be given for the first 4 weeks, in the morning with a meal. Following a favorable review of all safety data for each subject by the investigator, tesofensine 0.125 mg+metoprolol 25 mg will be given for the final 9 weeks (the above proposed dosing plan can be adjusted by the DSMB, if needed)

or

Arm 2) Placebo tablets matching tesofensine+metoprolol administered once a day, in the morning with a meal.

If, following the increase to 0.125/25 mg, the IMP is not tolerated; the investigator can reduce the dose back to 0.0625/25 mg, or stop the IMP completely.

Each tablet will be formulated separately; a currently available commercial formulation of extended-release metoprolol will be used.

### Example 3. Phase 2a Clinical Trial of Tesomet in Adult Patients with PWS

The anti-hyperphagia and weight reduction effect of Tesofensine in combination with Metoprolol (Tesomet) on PWS was investigated in a double-blind, randomized (2:1), placebo-controlled, multiple-dose, two-center, safety and efficacy study of co-administration of tesofensine/metoprolol in subjects with Prader-Willi syndrome. Example 3 concerns further observations from the clinical study of Example 1.

Subjects

Subjects with PWS (confirmed by genetic diagnosis) were 18-30 years of age with Body Mass Index (BMI) ≥25 kg/m², normal BP, normal lipid profile, and on a stable dose of GH for more than 2 months. Exclusion criteria included BP ≥140/90 mmHg, HR≥90, <50 bpm, Type 1 diabetes, diagnosis of personality or major depressive disorder, treatment with calcium channel or beta blockers, untreated hypo- or hyperthyroidism, bulimia or anorexia nervosa, and more than 5% weight loss within the last three months.

TABLE 7
Demographics, treatment and disposition.

| | | | | | | Completed |
|---|---|---|---|---|---|---|
| | Randomization | | Age, | Weight, | Treatment | study (to day |
| Country | number | Sex | yr | Kg | allocation | 91) |
| CZ | 101 | F | 28 | 115.8 | IMP | No |
| CZ | 102 | F | 20 | 86.4 | IMP | Yes |
| CZ | 103 | M | 26 | 94.1 | IMP | No |
| CZ | 104 | F | 26 | 82.9 | Placebo | No |
| HU | 201 | F | 27 | 162.9 | Placebo | Yes |
| HU | 202 | F | 18 | 67.5 | IMP | No |
| HU | 203 | M | 22 | 150 | Placebo | Yes |
| HU | 204 | F | 19 | 72 | IMP | No |
| HU | 205 | M | 19 | 124.4 | IMP | Yes |

CZ: Czech Republic,
HU: Hungary

The term “IMP” stands for Investigation Medicinal Product and corresponds to the co-administration of tesofensine (0.50 mg)/metoprolol (50 mg).

Methodology

The study included two arms: Tesomet 0.5/50 (combination of 0.50 mg tesofensine and 50 mg of metoprolol ER) or placebo tablets matching tesofensine+metoprolol succinate, each administered once daily in the morning with a meal.

The primary endpoint was change in body weight following 91 days of treatment with either tesofensine+metoprolol or placebo. Patients were weighed throughout the study period to track changes in body weight. Secondary endpoints included change in hyperphagia-related behavior, pharmacokinetic profile, effects of co-administration of tesofensine/metoprolol on HR, BP, glycemic control, lipid profile and body composition, and safety and tolerability.

Hyperphagia was measured using the Hyperphagia Questionnaire for Clinical Trials (HQ-CT), a validated hyperphagia questionnaire for clinical trials in patients with PWS (Crawford et al. 2015, ‘The International Development of The Modified Hyperphagia Questionnaire’, Value Health, 18: A761).

Results

Nine patients (three males, six females) were enrolled in the study, three of whom received placebo and six of whom received the combination treatment, Tesomet (Table 7).

Of the nine randomized subjects, four (two on Tesomet 0.5/50 and two on placebo) completed the study to the end (day 91). Five Tesomet 0.5/50 treated patients and two placebo treated patients completed eight weeks (56 days) of the study prior to withdrawal (Table 8).

TABLE 8
Number of Tesomet and placebo patients per visit.

| Treat- | Day | | | | | | | | | | | | | | |
|---|---|---|---|---|---|---|---|---|---|---|---|---|---|---|---|
| ment | 0 | 7 | 14 | 21 | 28 | 35 | 42 | 49 | 56 | 63 | 70 | 77 | 84 | 91 | 105 |
| Placebo | 3 | 3 | 3 | 2 | 2 | 1 | 3 | 2 | 2 | 2 | 2 | 2 | 2 | 2 | 2 |
| Tesomet | 6 | 6 | 6 | 6 | 6 | 5 | 5 | 5 | 5 | 4 | 2 | 2 | 2 | 2 | 2 |
| 0.5/50 | | | | | | | | | | | | | | | |

Tesomet 0.5/50 = Combination treatment of tesofensine (0.5 mg) and metoprolol (50 mg)

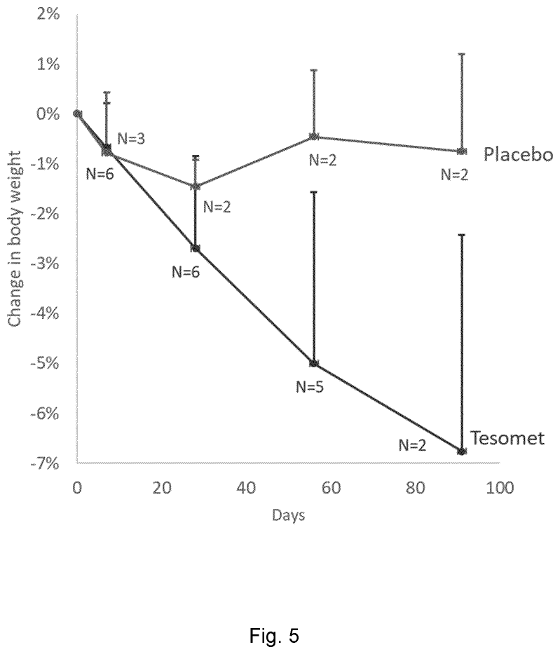

The mean change from baseline in body weight for patients receiving Tesomet 0.5/50 by Day 56 was −5.00% (n=5), compared to −0.46% (n=2) for placebo (FIG. 5).

At the end of the study, the mean change from baseline in body weight was −6.76% (n=2) for patients treated with Tesomet 0.5/50 and only −0.75% (n=2) for placebo treated patients (p=0.0466) (Table 9).

TABLE 9
Results for change in body weight.

| Treatment | | Visit 2 | Visit 5 | Visit 9 | Visit 14 |
|---|---|---|---|---|---|
| Placebo | N | 3 | 2 | 2 | 2 |
| | Mean (%) | −0.78 | −1.46 | −0.46 | −0.75 |
| | SEM (%) | 0.86 | 0.54 | 1.32 | 1.95 |
| Tesomet | n | 6 | 6 | 5 | 2 |
| 0.5/50 | Mean (%) | −0.67 | −2.69 | −5.00 | −6.76 |
| | SEM (%) | 0.4 | 0.83 | 1.72 | 4.33 |

N = number of patients; SEM = Standard; Error of the Mean; Visit 2 = day 7; Visit 5 = day 28; Visit 9 = day 56; Visit 14 = day 91

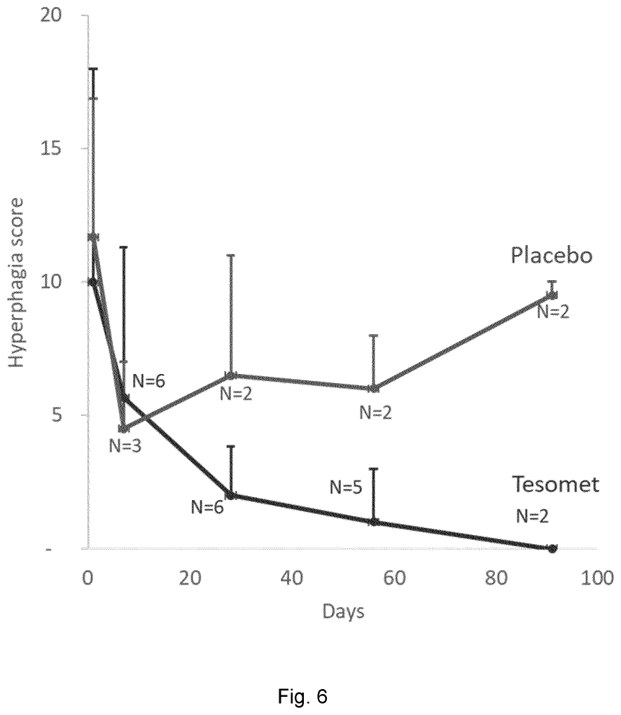

The results of the hyperphagia score showed that food cravings fell from 10 (n=6) at baseline to 1 (n=5) after 56 days and to 0 (n=2), or no observation of hyperphagia, after 91 days in patients on Tesomet 0.5/50 (FIG. 6). Hyperphagia scores for patients on placebo varied over time (n=2) but did not change from baseline to day 91.

The patients were treated with a dose based on distribution and metabolism as for healthy volunteers (combination of 0.50 mg tesofensine and 50 mg of metoprolol ER), but obtained a significant higher exposure than expected from the predictions. The plasma levels of tesofensine in this study were 20-40 ng/mL compared to efficacious target plasma levels of ˜10 ng/mL seen in previous studies in obese individuals at the same dosages.

Adverse events were reported in all patients participating in the trial. No serious adverse events (SAEs) were reported in the trial. The following psychiatric verbatim terms were reported: fatigue, behavior problems, depressive behavior, insomnia, fatigue, worsening (intensity and frequency) of intensive imagination and confabulation, hallucination, worsening of making things up, hallucination aggravated, screaming, crying, very angry behavior, destroying flower pot, screaming, crying, very angry behavior, shutting the door, not cooperative, emotional instability, strongly restless. In the six patients on active treatment there were 17 of these AEs and in the three subjects on placebo there were two of these AEs. The reported psychiatric AEs were suggestive of potential aggravation of pre-existing behavioral or CNS related dysfunctions in patients receiving Tesomet, and three of the AEs were classified as probably related to Tesomet treatment. Five of them led to early discontinuation. AEs were reversed after the completion of the study and in two cases where patients were offered a temporary reduction in dose during the study. Fifty-three percent of the AEs were mild, and the remaining were moderate in intensity.

Conclusions

Treatment with Tesomet demonstrated an encouraging efficacy signal, particularly the effect on hyperphagia is considered clinically meaningful, thus showing a promise for further investigation in patients with PWS; however, at a lower dose to ensure favorable benefit-risk profile.

### Example 4. Phase 2a Clinical Trial of Tesomet in Adolescent Patients with PWS

This study included a double blind (DB) part (3 months) and two Open Label Extensions (OLE), each 3 months. Adolescents (12 to 17 years of age) received: tesofensine 0.125 mg daily (0.25 mg every second day)+metoprolol succinate 25 mg. Example 4 relates to Example 2.

The initial double blind (DB) phase of this study utilized a dose of tesofensine of 0.125 mg daily. In the first 3-month Open Label Extension (OLE1), a dose of 0.125 mg tesofensine was maintained. In the second OLE (OLE2), the tesofensine dose, if judged safe by the principle investigator (PI), was increased to 0.25 mg daily.

Results

All nine of the randomized subjects in the adolescent study (five on Tesomet and four on placebo) completed the double blind part study to the end (day 91) (Table 10).

TABLE 10
Number of Tesomet and placebo patients per visit.

| Treat- | Day | | | | | | | | | | | | | | |
|---|---|---|---|---|---|---|---|---|---|---|---|---|---|---|---|
| ment | 0 | 7 | 14 | 21 | 28 | 35 | 42 | 49 | 56 | 63 | 70 | 77 | 84 | 91 | 105 |
| Placebo | 4 | 4 | 4 | 4 | 4 | 4 | 4 | 4 | 4 | 4 | 4 | 4 | 2 | 4 | 0 |
| Tesomet | 5 | 5 | 5 | 4 | 5 | 6 | 5 | 5 | 5 | 5 | 5 | 5 | 3 | 5 | 0 |
| 0.125/25 | | | | | | | | | | | | | | | |

Tesomet 0.125/25 = Combination treatment of tesofensine (0.125 mg daily) and metoprolol (25 mg daily)

In order to reduce the risk of adverse events that were observed in adults (Example 3) and believed to be driven by unexpectedly high plasma levels of tesofensine, the initial double blind (DB) phase of this study in adolescents utilized a significantly reduced dose of tesofensine of 0.125 mg daily, yielding plasma levels of 3-5 ng/mL. This can be compared with the of 20-40 ng/mL observed plasma concentration of tesofensine in the adult population treated with 0.50 mg tesofensine and 50 mg metoprolol in Example 3.

In the first 3-month Open Label Extension (OLE1), a dose of 0.125 mg tesofensine was maintained. In the second OLE (OLE2), the tesofensine dose, if judged safe by the principle investigator (PI), was increased to 0.25 mg daily with the aim of reaching a plasma concentration around 10 ng/mL, which has previously been demonstrated as efficacious in obese individuals at the same dosages. It was indeed found that the three patients completing OLE2 at 0.25 mg tesofensine did reach tesofensine plasma levels around 7-9 ng/mL.

In contrast to the adult study, both treatment and placebo groups in the adolescent study showed a significant weight gain (˜3%) over the 3 months in the DB treatment period. This weight gain could partly be accounted for by an increase in height of the patients.

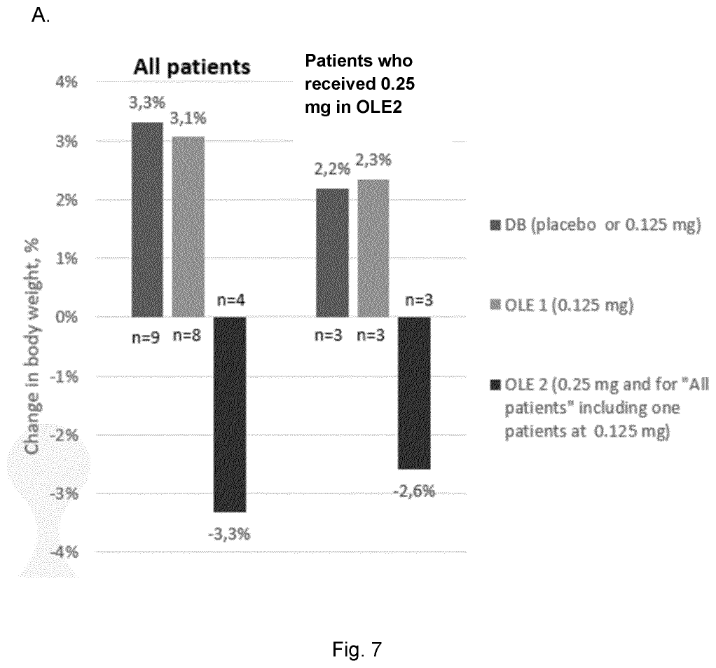

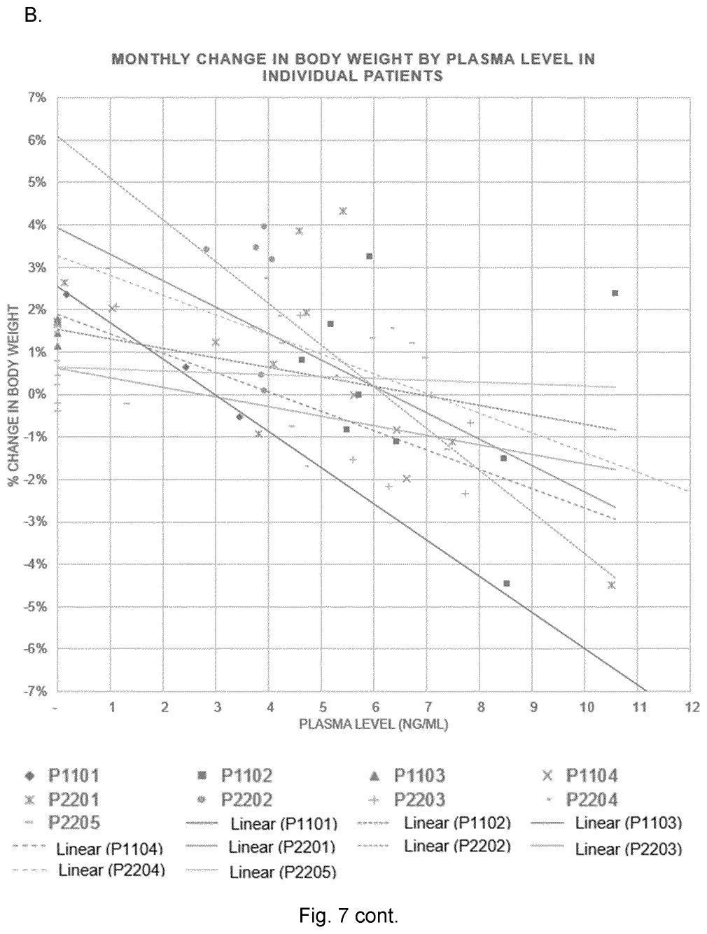

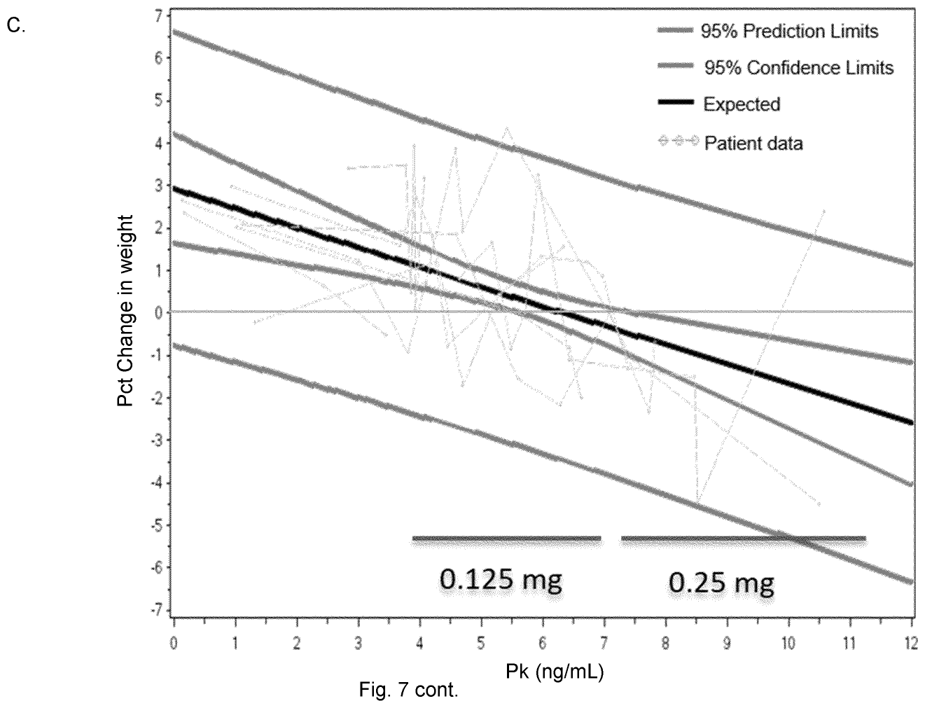

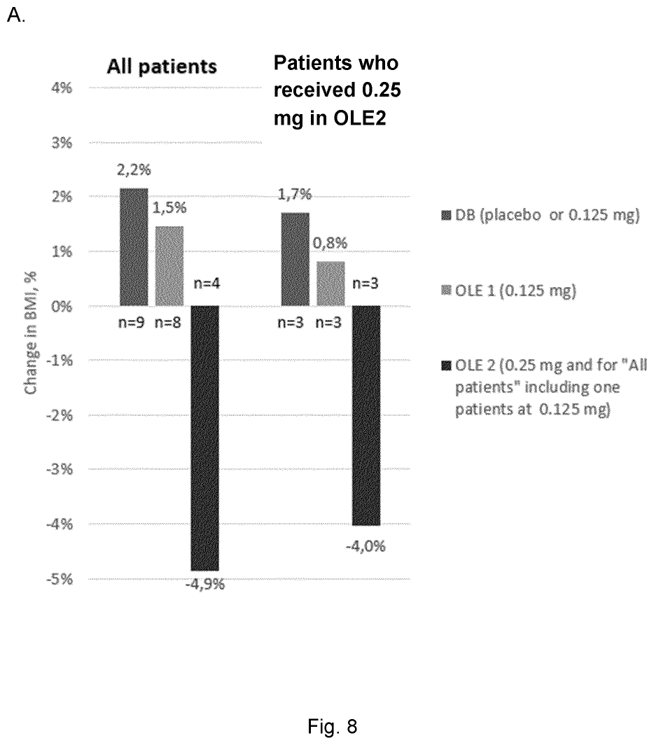

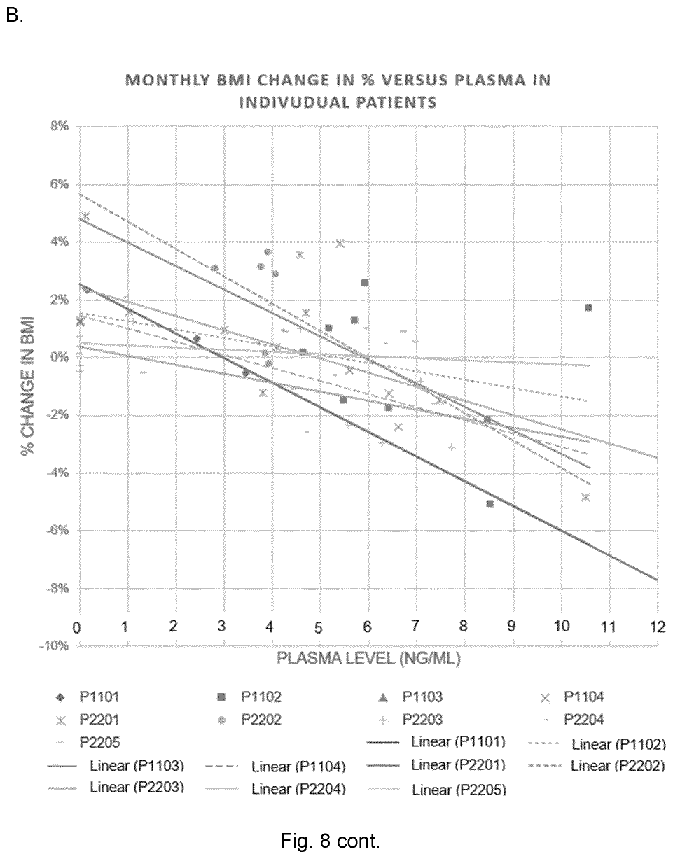

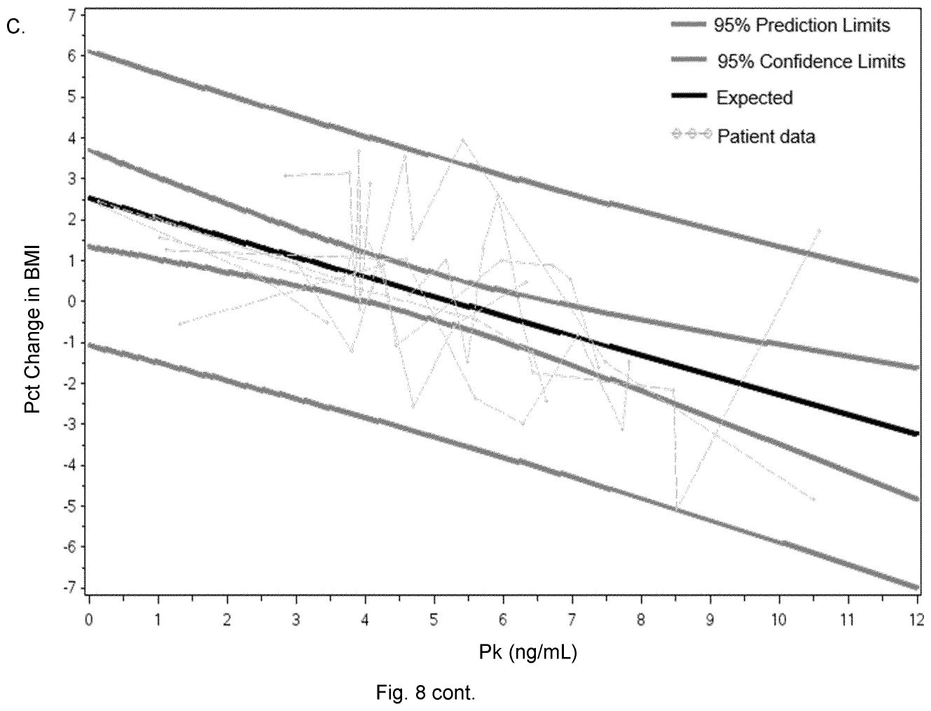

No difference compared to placebo was observed in the DB phase of the adolescent study with the 0.125 mg tesofensine dose in relation to weight change and hyperphagia (FIGS. 7 and 8, respectively). However, exploratory analysis of the two open label extensions (OLE1 and OLE2) suggest a strong correlation between plasma level of tesofensine and effect on monthly body weight change (FIG. 7). In FIG. 7A, all monthly visits with corresponding weight and PK measurements are plotted as single dots representing tesofensine plasma concentrations at the visit against body weight change since last monthly visit. Each point was fitted to linear regression lines for each patient and it is seen that for essentially all patients regression lines tended to point to a negative correlation between plasma concentration and monthly weight change. When each patient was analyzed first independently and then combined as a group (random coefficients analysis), a linear correlation line for the entire dataset was constructed (FIG. 7B).

In the random coefficients analysis, each subject was considered as having their own profile of data, i.e. their own set of weight change/hyperphagia values and corresponding pk values. The analysis was essentially conducted by fitting a regression line to each subject individually:

weightΔij=αj+βj·pkij

where j denotes subjects, j=1 to m, and i denotes the measurement with subject j, i=1 to nj. The slope βj and intercept αj estimate for each subject j=1 to m, along with their standard errors, were then obtained. In a final step, the βj and αj values were averaged across the m subjects to give the overall mean regression slope and mean intercept, βj and αj, which gives the correlation line, as well as the associated confidence interval.

This analysis shows a strong correlation between tesofensine plasma level and monthly weight loss with a slope significantly different from placebo (black line: p=0.005 on slope different from 0). The analysis also suggests that obtaining a tesofensine plasma level of around 6 ng/mL would counteract the observed baseline weight gain of around 3% and that higher plasma levels than 6 ng/mL which was achieved at 0.25 mg tesofensine would lead to a factual decrease in weight e.g. around 2% at 10 ng/mL tesofensine. 95% confidence limits and 95% prediction limits are shown in FIG. 7B as grey lines.

The weight gain of the adolescents could partly be accounted for by an increase in height. Thus, the change in Body Mass Index (BMI) was also considered (FIG. 8), and random coefficient analysis was conducted. The BMI values follow the same pattern as body weight. However, it can be noted that the improvement occurs at slightly lower plasma level than body weight.

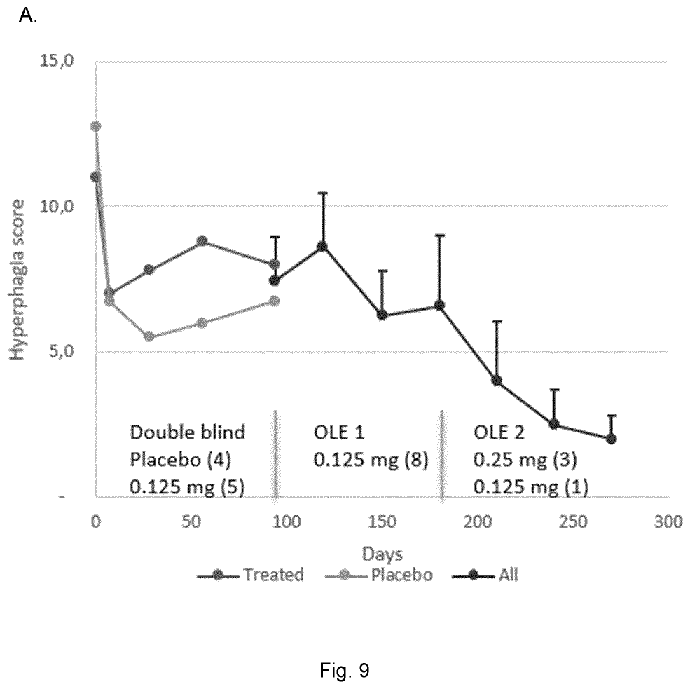

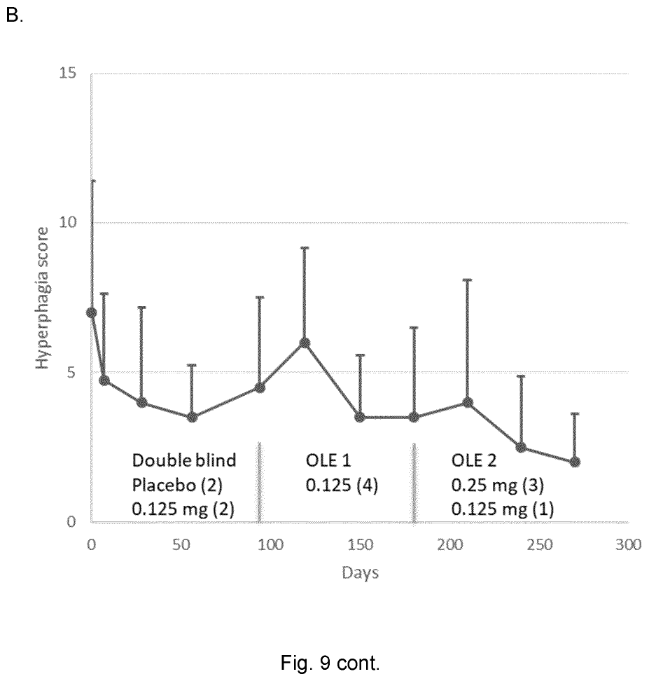

The hyperphagia scores did not differ from placebo during the DB part and OLE1 (tesofensine doses of 0.125 mg daily corresponding to plasma levels of 3-5 ng/mL tesofensine), It was however observed that hyperphagia scores dropped to single digit numerical values during OLE2 when 0.25 mg doses of tesofensine was introduced, leading to plasma levels around target levels of 10 ng/mL tesofensine (FIG. 9).

The increase in dose to 0.25 mg daily did not give rise to an increased rate of drug related adverse events. Effect of Tesomet on heart rate, diastolic and systolic blood pressure did not differ significantly from placebo in the DB part and no trends for change in the open label extensions were observed when compared to the DB part and follow up visit.

Conclusions

This study demonstrates that obtaining a tesofensine plasma level of around 6 ng/mL counteracts the observed baseline weight gain of around 3%. Plasma levels higher than 6 ng/mL, which was achieved at 0.25 mg tesofensine, lead to a factual decrease in weight, e.g. around 2% at 10 ng/mL tesofensine.

1. A method for treating hyperphagia in a Prader-Willi patient, comprising administering to the Prader-Willi patient a pharmaceutical composition comprising Tesofensine at a daily dosage of 0.01-0.250 mg Tesofensine.

2. (canceled)

3. The method according to claim 1, wherein the dosage results in a Tesofensine plasma or serum concentration of 5 to 15 ng/mL at steady state.

4-10. (canceled)

11. The method according to claim 1, wherein the dosage is about 0.25 mg Tesofensine.

12. The method according to claim 1, wherein the Prader-Willi patient is adolescent.

13-14. (canceled)

15. The method according to claim 1, wherein the pharmaceutical composition is administered in combination with metoprolol or a pharmaceutically acceptable salt thereof, or wherein the pharmaceutical composition further comprises metoprolol or a pharmaceutically acceptable salt thereof.

16-25. (canceled)

26. The method according to claim 15, wherein the pharmaceutical composition comprises:
a. a first composition comprising an extended release (ER) composition of an active pharmaceutical ingredient (API) that is metoprolol or a pharmaceutically acceptable salt thereof,
b. a second composition comprising an active pharmaceutical ingredient (API) that is Tesofensine or a pharmaceutically acceptable salt thereof, and
c. a third composition comprising an immediate release (IR) composition of an active pharmaceutical ingredient (API) that is metoprolol or a pharmaceutically acceptable salt thereof.

27. The method according to claim 15, wherein the daily dosage of metoprolol, or a pharmaceutically acceptable salt thereof, is 5 to 50 mg.

28-40. (canceled)

41. A pharmaceutical composition comprising 0.025 to 0.150 mg of Tesofensine, or a pharmaceutically acceptable salt thereof, and 5 to 25 mg Metoprolol, or a pharmaceutically acceptable salt thereof.

42. The pharmaceutical composition according to claim 41, wherein Metoprolol comprises an extended release composition of Metoprolol, or a pharmaceutically acceptable salt thereof, (ER Metoprolol); and an immediate release composition of Metoprolol, or a pharmaceutically acceptable salt thereof (IR Metoprolol).

43-47. (canceled)

48. The pharmaceutical composition according to claim 41, wherein the amount of Tesofensine, or a pharmaceutically acceptable salt thereof, is 0.125 mg.

49-59. (canceled)

60. The pharmaceutical composition according to claim 41, wherein the composition is in the form of a tablet or a capsule.

61-66. (canceled)

67. A method for reducing or maintaining of body weight in a Prader-Willi patient, comprising administering to the Prader-Willi patient a pharmaceutical composition comprising Tesofensine at a daily dosage of 0.01 to 0.250 mg Tesofensine.

68. The method according to claim 67, wherein the dosage results in a Tesofensine plasma or serum concentration of 5 to 15 ng/mL at steady state.

69. The method according to claim 67, wherein the dosage is about 0.125 mg Tesofensine.

70. The method according to claim 67, wherein the dosage is about 0.25 mg Tesofensine.

71. The method according to claim 67, wherein the Prader-Willi patient is adolescent.

72. The method according to claim 67, wherein the pharmaceutical composition is administered in combination with metoprolol or a pharmaceutically acceptable salt thereof, or wherein the pharmaceutical composition further comprises metoprolol or a pharmaceutically acceptable salt thereof.

73. The method according to claim 72, wherein the pharmaceutical composition comprises:
a. a first composition comprising an extended release (ER) composition of an active pharmaceutical ingredient (API) that is metoprolol or a pharmaceutically acceptable salt thereof,
b. a second composition comprising an active pharmaceutical ingredient (API) that is Tesofensine or a pharmaceutically acceptable salt thereof, and
c. a third composition comprising an immediate release (IR) composition of an active pharmaceutical ingredient (API) that is metoprolol or a pharmaceutically acceptable salt thereof.

74. The method according to claim 72, wherein the daily dosage of metoprolol, or a pharmaceutically acceptable salt thereof, is 5 to 50 mg.

75. The method according to claim 67, wherein the Body Mass Index (BMI) of the Prader-Willi patient is reduced by at least 2% after two months of treatment.
